SEQUENCE LISTING (57 sequences)

SEQ ID NO: 1 - DNA, 408 bp, synthetic construct
  source 1..408: mol_type = other DNA
atgagcaaaagcaatgaaccgggtaaagcaaccggtgaaggtaaaccggttaataacaaatggctgaacaatgccggtaaagatctgggtagtccggttccggatcgtattgcaaataaactgcgtgataaagaattcgagagcttcgatgattttcgtgaaaccttttgggaagaagttagcaaagatcctgaactgagcaaacagtttagccgcaataacaatgatcgtatgaaagttggtaaagcaccgaaaacacgtacccaggatgttagcggtaaacgtacctcatttgaactgaatcatcagaaaccgattgaacagaatggtggcgtttatgatatggataacattagcgttgttaccccgaaacgcaacattgatattgaaggcggtggtggtggttct

SEQ ID NO: 2 - DNA, 24 bp, synthetic construct
  source 1..24: mol_type = other DNA
ttggaggttttgttccagggtcca

SEQ ID NO: 3 - DNA, 135 bp, synthetic construct
  source 1..135: mol_type = other DNA
cttaaagaagctaaagagaaagcgatcgaagaattgaaaaaggctggcattacgtccgattattattttgatttgattaacaaggcaaagaccgtcgagggagtcaatgcccttaaagatgaaatcttaaaagct

SEQ ID NO: 4 - DNA, 50 bp, synthetic construct
  source 1..50: mol_type = other DNA
gatcaggtggaggaggttcgggcggtggtgggagcgggggtggcggttct

SEQ ID NO: 5 - DNA, 69 bp, synthetic construct
  source 1..69: mol_type = other DNA
ccgaaaaagaaacggaaagttgatcctaaaaagaagcgtaaggtagatccaaagaaaaaacgcaaagtt

SEQ ID NO: 6 - DNA, 51 bp, synthetic construct
  source 1..51: mol_type = other DNA
ggctcgggtggaggcggttcgggcgggggtggctctgggggtggcggatct

SEQ ID NO: 7 - DNA, 4104 bp, synthetic construct
  source 1..4104: mol_type = other DNA
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgcattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgac

SEQ ID NO: 8 - DNA, 51 bp, synthetic construct
  source 1..51: mol_type = other DNA
ggaagcggcggagggggcagtggcgggggtggaagtggtggaggtggcagc

SEQ ID NO: 9 - DNA, 69 bp, synthetic construct
  source 1..69: mol_type = other DNA
ccgaagaaaaaacgtaaagtagaccctaagaaaaaacgcaaggtagaccctaagaaaaagcggaaagtc

SEQ ID NO: 10 - DNA, 51 bp, synthetic construct
  source 1..51: mol_type = other DNA
ggatcaggtggaggaggttcgggcggtggtgggagcgggggtggcggttct

SEQ ID NO: 11 - DNA, 134 bp, synthetic construct
  source 1..134: mol_type = other DNA
ttaaagaagctaaagagaaagcgatcgaagaattgaaaaaggctggcattacgtccgattattattttgatttgattaacaaggcaaagaccgtcgagggagtcaatgcccttaaagatgaaatcttaaaagct

SEQ ID NO: 12 - DNA, 45 bp, synthetic construct
  source 1..45: mol_type = other DNA
ggtggaggaggttcgggcggtggtgggagcgggggtggcggttct

SEQ ID NO: 13 - DNA, 18 bp, synthetic construct
  source 1..18: mol_type = other DNA
catcaccaccaccatcac

SEQ ID NO: 14 - DNA, 18 bp, synthetic construct
  source 1..18: mol_type = other DNA
ttcggcttctggcgtgtg

SEQ ID NO: 15 - DNA, 44 bp, synthetic construct
  source 1..44: mol_type = other DNA
agctgaccggcagcaaaattgctttgatgacctcctcgcccttg

SEQ ID NO: 16 - DNA, 44 bp, synthetic construct
  source 1..44: mol_type = other DNA
agctgaccggcagcaaaattgcctctgctaaccatgttcatgcc

SEQ ID NO: 17 - DNA, 17 bp, synthetic construct
  source 1..17: mol_type = other DNA
cctcctcgcccttgctc

SEQ ID NO: 18 - DNA, 43 bp, synthetic construct
  source 1..43: mol_type = other DNA
agctgaccggcagcaaaattgctgggcaacgtgctggttattg

SEQ ID NO: 19 - DNA, 17 bp, synthetic construct
  source 1..17: mol_type = other DNA
cctcctcgcccttgctc

SEQ ID NO: 20 - protein, 45 aa, synthetic construct
LKEAKEKAIEELKKAGITSDYYFDLINKAKTVEGVNALKDEILKA

SEQ ID NO: 21 - protein, 7 aa, synthetic construct
PKKKRKV

SEQ ID NO: 22 - protein, 15 aa, synthetic construct
GGGGSGGGGSGGGGS

SEQ ID NO: 23 - protein, 1439 aa, synthetic construct
GPPKKKRKVDPKKKRKVDPKKKRKVMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVDPKKKRKVDPKKKRKVGSGGGGSGGGGSGGGGSHHHHHH

SEQ ID NO: 24 - protein, 1561 aa, synthetic construct
GPLKEAKEKAIEELKKAGITSDYYFDLINKAKTVEGVNALKDEILKAGSGGGGSGGGGSGGGGSPKKKRKVDPKKKRKVDPKKKRKVMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVDPKKKRKVDPKKKRKVGSGGGGSGGGGSGGGGSLKEAKEKAIEELKKAGITSDYYFDLINKAKTVEGVNALKDEILKAGGGGSGGGGSGGGGSHHHHHH

SEQ ID NO: 25 - protein, 1501 aa, synthetic construct
GPLKEAKEKAIEELKKAGITSDYYFDLINKAKTVEGVNALKDEILKAGSGGGGSGGGGSGGGGSPKKKRKVDPKKKRKVDPKKKRKVMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVDPKKKRKVDPKKKRKVGSGGGGSGGGGSGGGGSHHHHHH

SEQ ID NO: 26 - protein, 1591 aa, synthetic construct
GPPKKKRKVDPKKKRKVDPKKKRKVGGGGSGGGGSGGGGSLKEAKEKAIEELKKAGITSDYYFDLINKAKTVEGVNALKDEILKAGSGGGGSGGGGSGGGGSMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSGGGGSGGGGSGGGGSLKEAKEKAIEELKKAGITSDYYFDLINKAKTVEGVNALKDEILKAGGGGSGGGGSGGGGSPKKKRKVDPKKKRKVDPKKKRKVGGGGSGGGGSGGGGSHHHHHH

SEQ ID NO: 27 - protein, 1497 aa, synthetic construct
GPPKKKRKVDPKKKRKVDPKKKRKVGGGGSGGGGSGGGGSLKEAKEKAIEELKKAGITSDYYFDLINKAKTVEGVNALKDEILKAMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVDPKKKRKVDPKKKRKVGGGGSGGGGSGGGGSHHHHHH

SEQ ID NO: 28 - protein, 1484 aa, synthetic construct
GPPKKKRKVDPKKKRKVDPKKKRKVMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVDPKKKRKVDPKKKRKVGSGGGGSGGGGSGGGGSLKEAKEKAIEELKKAGITSDYYFDLINKAKTVEGVNALKDEILKAHHHHHH

SEQ ID NO: 29 - protein, 1368 aa, synthetic construct [sequence omitted]

SEQ ID NO: 30 - protein, 46 aa, synthetic construct
LAEAKVLANRELDKYGVSDYYKNLINNAKTVEGVKALIDEILAALP

SEQ ID NO: 31 - protein, 47 aa, synthetic construct
LKNAKEDAIAELKKAGITSDFYFNAINKAKTVEEVNALKNEILKAHA

SEQ ID NO: 32 - protein, 44 aa, synthetic construct
LAQAKEAAIKELKQYGIGDYYIKLINNAKTVEGVESLKNEILKA

SEQ ID NO: 33 - protein, 44 aa, synthetic construct
LAEAKVLALRELDKYGVSDYYKDLIDKAKTVEGVKALIDEILAA

SEQ ID NO: 34 - protein, 46 aa, synthetic construct
LAEAKVLANRELDKYGVSDFYKRLINKAKTVEGVEALKLHILAALP

SEQ ID NO: 35 - protein, 46 aa, synthetic construct
LAEAKEAANAELDSYGVSDFYKRLIDKAKTVEGVEALKDAILAALP

SEQ ID NO: 36 - protein, 16 aa, synthetic construct
KRPAATKKAGQAKKKK

SEQ ID NO: 37 - protein, 44 aa, synthetic construct
GRYLTQETNKVETYKEQPLKTPGKKKKGKPGKRKEQEKKKRRTR

SEQ ID NO: 38 - protein, 23 aa, synthetic construct
PKKKRKVDPKKKRKVDPKKKRKV

SEQ ID NO: 39 - protein, 8 aa, synthetic construct
  VARIANT 3: note = Lysine or Arginine
  VARIANT 6: note = Lysine or Arginine
KRXRKXRK

SEQ ID NO: 40 - skipped

SEQ ID NO: 41 - protein, 8 aa, synthetic construct
  VARIANT 3: note = Any amino acid
  VARIANT 4: note = Tryptophan, Phenylalanine, or Tyrosine
  VARIANT 5..6: note = Any amino acid
KRXXXXAF

SEQ ID NO: 42 - skipped

SEQ ID NO: 43 - protein, 8 aa, synthetic construct
  VARIANT 3: note = Lysine or Arginine
  VARIANT 6: note = Lysine or Arginine
KRXRKXRK

SEQ ID NO: 44 - skipped

SEQ ID NO: 45 - protein, 8 aa, synthetic construct
  VARIANT 3: note = Any amino acid
  VARIANT 4: note = Tryptophan, Phenylalanine, or Tyrosine
  VARIANT 5..6: note = Any amino acid
KRXXXXAF

SEQ ID NO: 46 - skipped

SEQ ID NO: 47 - protein, 8 aa, synthetic construct
GGGGGGGG

SEQ ID NO: 48 - protein, 6 aa, synthetic construct
GGGGGG

SEQ ID NO: 49 - protein, 18 aa, synthetic construct
KESGSVSSEQLAQFRSLD

SEQ ID NO: 50 - protein, 14 aa, synthetic construct
EGKSSGSGSESKST

SEQ ID NO: 51 - protein, 12 aa, synthetic construct
GSAGSAAGSGEF

SEQ ID NO: 52 - protein, 5 aa, synthetic construct
  REPEAT 1..5: note = Repeats 1 to 3 times
EAAAK

SEQ ID NO: 53 - protein, 46 aa, synthetic construct
AEAAAKEAAAKEAAAKEAAAKALEAEAAAKEAAAKEAAAKEAAAKA

SEQ ID NO: 54 - protein, 5 aa, synthetic construct
PAPAP

SEQ ID NO: 55 - protein, 12 aa, synthetic construct
AEAAAKEAAAKA

SEQ ID NO: 56 - protein, 17 aa, synthetic construct
GGGGSSPLGLWAGGGGS

SEQ ID NO: 57 - protein, 12 aa, synthetic construct
TRHRQPRGWEQL

(19) United States
(12) Patent Application Publication
(10) Pub. No.: US 2026/0234611 A1
(43) Pub. Date: Aug. 13, 2026

(54) ALBUMIN BINDING RECOMBINANT CAS9 PROTEINS AND USES THEREOF

(71) Applicant: Vanderbilt University, Nashville, TN (US)

(72) Inventors: Craig L. Duvall, Nashville, TN (US); Ronald Brock Fletcher, Nashville, TN (US); Isabel Vallecillo-Viejo, Nashville, TN (US); Larry D. Stokes, Nashville, TN (US)

(21) Appl. No.: 19/473,336

(22) PCT Filed: Apr. 5, 2024
(86) PCT No.: PCT/US2024/023457

§ 371 Date: Oct. 7, 2025

Related U.S. Application Data
(60) Provisional application No. 63/494,911, filed on Apr. 7, 2023.

(51) Int. Cl. C12N 15/11; C12N 9/22

(52) U.S. Cl. CPC C12N 15/111; C12N 9/22; C12N 9/222; C12N 15/11; C12N 2310/20

(57) ABSTRACT

Disclosed herein are recombinant proteins that can take advantage of binding albumin to improve gene editing applications. An example recombinant protein includes an albumin binding domain, a nuclear localization domain, and a Cas9 domain. Also disclosed are compositions including the recombinant protein and, e.g., one or more guide RNA, and methods of using the recombinant protein and compositions thereof.

### CROSS-REFERENCE TO RELATED APPLICATIONS

This application claims priority to U.S. Provisional Patent Application No. 63/494,911 filed on Apr. 7, 2023, which is incorporated fully herein by reference.

### STATEMENT REGARDING FEDERALLY SPONSORED RESEARCH

This invention was made with government support under Grant Nos. R38HL143619 and R01CA260958-02S1 awarded by the National Institutes of Health. The government has certain rights in the invention.

### INTRODUCTION

Manipulating genetic information for correcting disease-causing mutations has recently emerged as a translationally relevant therapeutic approach. In particular, the CRISPR-Cas9 system has shown promise for correction of diseases including sickle-cell disease, thalassemias, cystic fibrosis, primary immunodeficiencies, Duchenne muscular dystrophy, and cancer. Despite the promise of gene editing for therapeutic purposes, the potential of gene editing systems is limited by the lack of safe and effective delivery technologies.

### BRIEF SUMMARY OF THE DISCLOSURE

The present disclosure concerns methods and compositions for the correction and/or treatment of one or more genetic diseases. In preferred embodiments, the genetic disease is selected from any one of Duchenne muscular dystrophy (DMD), Limb Girdle muscular dystrophy (LGMD), Charcot-Marie Tooth Syndrome (CMT), Myotonic Dystrophy, Huntington's Disease, and Spinal Cerebellar Ataxia.

In one aspect, the present disclosure pertains to a recombinant protein comprising an albumin binding domain; a nuclear localization domain; and a Cas9 domain. In some embodiments, the recombinant protein further comprises a linker between the albumin binding domain and the nuclear localization domain. In some embodiments, the recombinant protein includes more than one albumin binding domain. In some embodiments, the recombinant protein includes more than one nuclear localization domain. In some embodiments, the recombinant protein includes a combination of more than one albumin binding domain and more than one nuclear localization domain. In some embodiments, the recombinant protein comprises an amino acid sequence that is at least 95% identical to any one of SEQ ID NO:24, SEQ ID NO: 25, SEQ ID NO:26, SEQ ID NO:27, and SEQ ID NO:28. In some embodiments, the recombinant protein comprises an amino acid sequence that is at least 99% identical to any one of SEQ ID NO:24, SEQ ID NO:25, SEQ ID NO:26, SEQ ID NO:27, and SEQ ID NO:28. In some embodiments, the recombinant protein comprises, in a N-terminus to a C-terminus direction: the nuclear localization domain, the linker, the albumin binding domain, and the Cas9 domain. In some embodiments, the recombinant protein comprises an amino acid sequence that is at least 95% identical to any one of SEQ ID NO:26 and SEQ ID NO:27. In some embodiments, the recombinant protein comprises an amino acid sequence that is at least 99% identical to any one of SEQ ID NO:26 and SEQ ID NO:27.

In one aspect, the present disclosure pertains to a method of modifying a targeted gene associated with a disease in a patient, the method comprising administering to the patient a therapeutically effective amount of a composition, the composition comprising a recombinant protein and one or more guide RNAs. In some embodiments, the targeted gene is selected from any one of a dystrophin (DMD) gene, a calpain 3 (CAPN3) gene, a dysferlin (DYSF) gene, an alpha-sarcoglycan (SGCA) gene, a beta-sarcoglycan (SGCB) gene, a gamma-sarcoglycan (SGCG) gene, a delta-sarcoglycan gene (SGCD) gene, a telethonin (TCAP) gene, a fukutin-related protein (FKRP) gene, an anoctamin 5 (ANO5) gene, a DM1 gene, a DM2 gene, a neurotrophin 3 (NT3) gene, and a huntingtin (HTT) gene. In some embodiments, the targeted gene is a human gene.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1A: Recombinant spCas9 proteins retain enzymatic activity. (A) Schematic of example ABD-Cas-9 recombinant proteins. NLS: SV40 nuclear localization signal, L: GGGSx3 linker, ABD: albumin binding domain, Cas9: SpCas9, and His: 6×histidine tag.

FIG. 1B: Agarose gel electrophoresis showing Ai9 DNA plasmid cleavage using N-ABD-spCas9-internal protein (i). This assay measures the cell-free cleavage of an Ai9 plasmid by SpCas9 in the presence of different engineered proteins. Assay was run in the presence of 0, 1, or 10× molar ratio of human serum albumin (HSA). (ii) Quantification of plasmid site targeted cutting efficiency by densitometry (n=3).

FIG. 2A. In vitro binding responses of example recombinant Cas9 protein, NC-ABD-Internal, to HSA at different concentrations using biolayer interferometry (BLI).

FIG. 2B: In vitro binding responses of example recombinant Cas9 protein, N-ABD-Internal, to HSA at different concentrations using BLI.

FIG. 2C: In vitro binding responses of example recombinant Cas9 protein, NC-ABD-External, to HSA at different concentrations using BLI.

FIG. 2D: In vitro binding responses of example recombinant Cas9 protein, N-ABD-External, to HSA at different concentrations using BLI.

FIG. 2E: Table showing the binding affinity (Kp) of example proteins to albumin.

FIG. 3A: Schematic of experimental scheme for example recombinant spCas9 proteins in vivo editing upon local injection.

FIG. 3B: Representative in vivo imaging system (IVIS) images (upper panel) and histology for example recombinant spCas9 proteins in vivo editing upon intramuscular (IM) injection. A pair (left and right) of tibialis anterior (TA) muscles are shown from a representative mouse from each treatment group. Fluorescent signal of tdTomato (Ai9 reporter turn-on) was quantified by IVIS in the explanted TA muscles as shown in FIG. 3C.

FIG. 3C: Quantification of editing efficiency of example recombinant spCas9 proteins in vivo editing upon local injection based on IVIS relative radiance efficiency.

FIG. 4A: IVIS images of mice injected intravenously (IV) with example recombinant spCas9 following BaCl₂ induced muscle injury.

FIG. 4B: Quantification of relative fluorescence for each example protein tested in different organs following BaCl₂ induced muscle injury.

FIG. 5A: Schematic of experimental scheme for example recombinant spCas9 proteins in vivo editing upon systemic injection.

FIG. 5B: Representative IVIS images of TA muscles for example recombinant spCas9 proteins in vivo gene editing upon systemic injection. Both TA muscles were injected with BaCl₂ to induce inflammation and injury prior to IV delivery of carrier-free ribonucleoproteins (RNPs). A pair (left and right) of TA muscles are shown from a representative mouse from each treatment group.

FIG. 5C: Quantification of in vivo editing efficiency after systemic injection of example recombinant spCas9 based on IVIS relative radiance efficiency (left panel) or Indel Detection by Amplicon Analysis (IDAA; right panel).

FIG. 6. Recombinant expression of Cas9 proteins using double purification with NINTA and Im7. SDS page of purification products. Lane 1: molecular weight marker, Lane 2: crude lysate, Lane 3: flow-through NINTA column, Lane 4: wash, Lane 5: elution with 150 mM imidazole, Lane 7: elution after Im7 purification for 6×NLSCas9, Lane 8: N-ABD-external Cas9 elusion, Lane 9: NC-ABD-external Cas9 elution, Lane 10: N-ABD-Cas9 internal elution, Lane 11: NC-ABD-Cas9 internal elution, and Lane 12: C-ABD-Cas9 external elution. All elution samples were passed through a protein concentrator with 100 kDa molecular weight cutoff. Red arrow indicates Cas9 product of correct size (e.g., 164 kDa).

FIG. 7. Schematic of example ABD-Cas-9 recombinant proteins.

### DETAILED DESCRIPTION OF THE DISCLOSURE

The present disclosure provides a strategy to systemically deliver a carrier-free spCas9 enzyme ribonucleoprotein (RNP) complex for CRISPR-mediated gene editing. The disclosed engineered forms of the spCas9 nuclease can be appended at different protein sites with, e.g., an albumin-binding domain (ABD) for increasing half-life and biodistribution to different tissues, such as inflamed muscle. A nuclear localization signal (NLS) can also be added to the proteins for nuclear entry and increased gene editing activity. Using the disclosed strategy, it was found that (1) ABD-spCas9 retains sequence targeted DNA cleavage activity and binds albumin in vitro, (2) ABD-spCas9 proteins show increased biodistribution to inflamed muscle after intravenous administration in mice, and (3) ABD-spCas9 proteins achieve significant gene editing in vivo following both intramuscular and intravenous administration in mice. It was further found that the addition of ABD at some sites reduces nuclease activity of the spCas9.

### 1. Definitions

Unless otherwise defined, all technical and scientific terms used herein have the same meaning as commonly understood by one of ordinary skill in the art. In case of conflict, the present document, including definitions, will control. The materials, methods, and examples disclosed herein are illustrative only and not intended to be limiting. Methods and materials similar or equivalent to those described herein can be used in practice or testing of the disclosed technology. All publications, patent applications, patents and other references mentioned herein are incorporated by reference in their entirety.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising.” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are contemplated, and for the range 1.5-2, the numbers 1.5, 1.6, 1.7, 1.8, 1.9, and 2 are contemplated.

The term “C-terminal end,” as used herein, refers to a fragment of a polypeptide that begins at any amino acid in the C-terminal half of the polypeptide and ends at the last amino acid of the polypeptide. The term “C-terminus,” as used herein, refers to the last amino acid of a polypeptide.

The term “Cas9,” as used herein, refers to a Type II RNA-guided CRISPR-Associated nuclease protein that is the active enzyme in a CRISPR-Cas9 system. In its active state, Cas9 is capable of introducing targeted double-stranded DNA breaks. “nCas9” refers to a Cas9 that has one of the two nuclease domains inactivated, i.e., either the RuvC or HNH domain. nCas9 is capable of cleaving only one strand of target DNA (a “nickase”). The term “dCas9” refers to a Cas9 that has both nuclease domains inactivated. dCas9 is incapable of cleaving target DNA but retains its ability to be guided by RNA to a particular genomic locus.

“Cas9” refers to both naturally occurring and recombinant Cas9 proteins. A wildtype Cas9 nuclease has two functional domains, e.g., RuvC and HNH, that cut different DNA strands. Cas9 enzymes described herein can comprise a HNH or HNH-like nuclease domain and/or a RuvC or RuvC-like nuclease domain. Cas9 can induce double-strand breaks in genomic DNA (e.g., a targeted gene) when both functional domains are active. The Cas9 enzyme can comprise one or more catalytic domains of a Cas9 protein derived from bacteria belonging to the group of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the two catalytic domains are derived from different bacteria species. In some embodiments, the Cas9 protein is derived from Streptococcus pyogenes (SpCas9).

The terms “complement” or “complementary,” as used herein, can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, NC.). The healthy or normal levels or ranges for a target or for a protein activity may be defined in accordance with standard practice. A control may be a subject or cell without a composition as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

The terms “Duchenne Muscular Dystrophy” or “DMD,” as used interchangeably herein, refer to a recessive, fatal, X-linked disorder that results in muscle degeneration and eventual death. DMD is a common hereditary monogenic disease and occurs in 1 in 3500 males and is the result of inherited or spontaneous mutations in the dmd gene that cause nonsense or frameshift mutations that affect expression of the resultant dystrophin protein. The majority of dystrophin mutations that cause DMD are deletions of exons that disrupt the reading frame and cause premature translation termination in the dystrophin gene. DMD patients typically lose the ability to physically support themselves during childhood or early adolescence and become progressively weaker throughout the teenage years before death in their twenties.

The term “genetic disease” as used herein refers to a disease, partially or completely, directly or indirectly, caused by one or more abnormalities in the genome, especially a condition that is present from birth. The abnormality may be a mutation, an insertion or a deletion. The abnormality may affect the coding sequence of the gene or its regulatory sequence. In one embodiment, this term expressly includes, but is not limited to DMD, LGMD, Huntington's disease, CMT, Myotonic Dystrophy and Spinal Cerebellar Ataxia.

“Genetic construct” as used herein refers to the DNA or RNA molecules that comprise a polynucleotide that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed. The regulatory elements may include, for example, a promoter, an enhancer, an initiation codon, a stop codon, or a polyadenylation signal.

The terms “genome editing” or “gene editing” as used herein refer to altering, regulating, or modifying a mutant gene (i.e., one encoding a truncated protein or non-functional protein) or an aberrant region of a gene, such that a full-length or partially full-length functional protein is expressed. Such activity may alternatively be considered “correcting” or “restoring” a mutant gene's functionality and may include replacing or excising an aberrant region of the mutant gene or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site, or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene, in some embodiments, may also include deleting a non-essential or aberrant gene segment by the simultaneous action of two nucleases on the same DNA strand. In some embodiments, genome editing may include knocking out a gene, such as a mutant gene or a normal gene. In some embodiments, genome editing may be used to treat disease or enhance muscle repair by regulating, modifying, or changing the gene of interest.

The terms “guide RNA” or “gRNA,” which may be used interchangeably herein, refer to one or more RNA molecules, preferably a synthetic RNA molecule, that comprise the RNA component of a CRISPR system (e.g., a CRISPR-Cas9 system) that guides a CRISPR-associated nuclease (e.g., Cas9) to a target polynucleotide or targeted gene. In one embodiment, a gRNA is comprised of a targeting sequence and scaffold sequence. In some embodiments, the gRNA is a single-guide RNA (sgRNA). In some embodiments, the sgRNA is composed of a crRNA and tracrRNA molecule. A sgRNA can be administered or formulated, e.g., as a synthetic RNA, or as a nucleic acid comprising a sequence encoding the gRNA, which is then expressed in one or more target cells. As would be evident to one of ordinary skill in the art, various tools may be used to design and/or optimize the sequence of a gRNA, for example, to increase the specificity and/or precision of genomic editing. In general, an ideal gRNA has a high predicted on-target efficiency and low off-target efficiency based on any of the available web-based tools. Candidate gRNAs may be further assessed by manual inspection and/or experimental screening. Examples of web-based tools include, without limitation, CRISPR seek, CRISPR Design Tool, Cas-OFFinder, E-CRISP, ChopChop, CasOT, CRISPR direct, CRISPOR, BREAKING-CAS, CrispRGold, and CCTop (Safari, et al. Current Pharma. Biotechol. (2017) 18 (13)). Such tools are also described, for example, in PCT Publication No. WO2014093701A1 and Liu, et al., “Computational approached for effective CRISPR guide RNA design and evaluation”, Comput Struct Biotechnol J., 2020; 18:35-44, each of which is incorporated by reference herein in its entirety for all purposes.

The term “heterologous” as used herein refers to nucleic acid comprising two or more subsequences that are not found in the same relationship to each other in nature. For instance, a nucleic acid that is recombinantly produced typically has two or more sequences from unrelated genes synthetically arranged to make a new functional nucleic acid, for example, a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. Similarly, a heterologous protein indicates that the protein comprises two or more subsequences that are not found in the same relationship to each other in nature (for example, a “fusion protein,” where the two subsequences are encoded by a single nucleic acid sequence).

The terms “homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including targeted addition of whole genes. If a donor template is provided along with a CRISPR-Cas9 gene editing system, then the cellular machinery will repair the break by homologous recombination, which is enhanced several orders of magnitude in the presence of DNA cleavage. When the homologous DNA piece is absent, non-homologous end joining may take place instead.

The terms “identity,” “identical,” “percent identity,” and/or “percent identical,” as used herein as applicable to one or more particular polynucleotide or amino acid sequences, refer to the proportion of identical residues between a particular reference sequence and another sequence, as calculated by a pairwise alignment using the Needleman-Wunsch algorithm using a generally available alignment program, e.g., the Needle (EMBOSS) program. In cases where the two sequences are of different lengths or the alignment produces one or more staggered ends and the specified region of comparison includes only a single sequence, the residues of the single sequence are included in the denominator but not the numerator for the purposes of calculating identity. When comparing DNA and RNA, thymine (T) and uracil (U) may be considered equivalent.

The terms “nucleic acid,” “oligonucleotide” or “polynucleotide” as used herein refer to deoxyribonucleotides or ribonucleotides and polymers thereof in either single- or double-stranded form and complements thereof. In some embodiments, the term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs). Any combinations of bases including uracil, adenine, thymine, cytosine, guanine, inosine, xanthine hypoxanthine, isocytosine and isoguanine are expressly contemplated by this application.

The term “N-terminal end,” as used herein, refers to a fragment of a polypeptide that begins at the first amino acid of the polypeptide and ends at any amino acid in the N-terminal half of the polypeptide. The term “N-terminus,” as used herein, refers to the first amino acid of a polypeptide.

A “peptide” or “polypeptide,” as used herein, refers to a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and can be 10 to 1,500 amino acids in length. Example domains include domains with enzymatic activity or ligand binding activity. Typical domains can be made up of sections of lesser organization such as stretches of beta-sheet and alpha-helices. “Tertiary structure” refers to the complete three-dimensional structure of a polypeptide. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif or of different motifs.

“Recombinant” when used with reference to, e.g., a cell, or nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed, or not expressed at all.

The terms “ribonucleoprotein” and “RNP,” as used herein interchangeably, refer to a heterogeneous complex containing both protein and RNA components. In preferred embodiments, the RNP is SpCas9 and an sgRNA.

The terms “subject” or “subject in need thereof,” as used herein, refer to a target of administration, which optionally displays symptoms related to a particular disease, pathological condition, disorder, or the like. The subject can be a vertebrate, such as a mammal, a fish, a bird, a reptile, or an amphibian. Thus, the subject can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

“Substantially identical” can mean that a first and second amino acid or polynucleotide sequence are at least 60%, 65%, 70%, 75%, 80%, 85%, 90%, 95%, 96%, 97%, 98%, or 99% identical over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, or 1100 amino acids or nucleotides, respectively.

The terms “target [ed] gene” or “target [ed] polynucleotide” as used herein refer to any nucleotide sequence encoding a known or putative gene product. The target gene may be a mutated gene involved in a genetic disease.

The terms “therapeutically effective amount” or “effective amount,” as used herein, refer to an amount sufficient to effect beneficial or desirable biological and/or clinical results.

The terms “treatment” or “treating,” as used herein, refer to the medical management of a patient with the intent to heal, cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Vector” as used herein means a nucleic acid sequence containing an origin of replication. A vector may be capable of directing the delivery or transfer of a polynucleotide sequence to target cells, where it can be replicated or expressed. A vector may contain an origin of replication, one or more regulatory elements, and/or one or more coding sequences. A vector may be a viral vector, bacteriophage, bacterial artificial chromosome, plasmid, cosmid, or yeast artificial chromosome. A vector may be a DNA or RNA vector. A vector may be a self-replicating extrachromosomal vector. Viral vectors include, but are not limited to, adenovirus vector, adeno-associated virus (AAV) vector, retrovirus vector, or lentivirus vector. A vector may be an adeno-associated virus (AAV) vector. The vector may encode a recombinant protein, at least one gRNA molecule, or both.

### 2. Recombinant Proteins

In one aspect, disclosed are recombinant proteins that pair the gene editing capabilities of Cas9 with the ability to bind albumin. The recombinant protein can include a Cas9 domain, an albumin binding domain, and a nuclear localization domain. The recombinant protein can also include a linker, e.g., between different domains. The disclosed recombinant protein can enable carrier-free genome editing via direct RNP delivery as a translatable therapeutic platform.

The domains can be positioned in a number of different ways within the recombinant protein. For example, the albumin binding domain can be located at the N-terminal end, C-terminal end, or both. The nuclear localization domain can be located at the N-terminal end, C-terminal end, or both. In addition, the albumin binding domain can be located internally, externally, or both in relation to the nuclear localization domain. In some embodiments, the albumin binding domain is between the Cas9 domain and the nuclear localization domain.

In some embodiments, the recombinant protein includes, in a N-terminus to a C-terminus direction: the nuclear localization domain; the linker; the albumin binding domain; and the Cas 9 domain.

In some embodiments, the recombinant protein includes, in a N-terminus to a C-terminus direction: the nuclear localization domain; the linker; the albumin binding domain; the Cas 9 domain; the albumin binding domain; the linker; and the nuclear localization domain.

In some embodiments, the recombinant protein includes a multi-domain structure of formula (I)

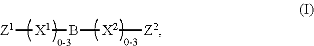

wherein: B is a Cas9 domain; X¹ and X², at each occurrence, are each independently a linker, an albumin binding domain, or a nuclear localization domain; Z¹ and Z² are each independently an albumin binding domain or a nuclear localization domain. In some embodiments, the multi-domain structure of formula (I) has at least one albumin binding domain and at least one nuclear localization domain.

In some embodiments, X¹ is a linker or an albumin binding domain; and X² is a linker or an albumin binding domain.

In some embodiments, X¹ is repeated two times and at each occurrence is independently a linker or an albumin binding domain. In some embodiments, X¹ is repeated three times and at each occurrence is independently a linker or an albumin binding domain.

In some embodiments, X² is not present. In some embodiments, X² is present one time and is a linker or an albumin binding domain. In some embodiments, X² is repeated two times and at each occurrence is independently a linker or an albumin binding domain. In some embodiments, X² is repeated three times and at each occurrence is independently a linker or an albumin binding domain.

In some embodiments, Z¹ is a nuclear localization domain; and Z² is a nuclear localization domain.

In some embodiments, X¹ is a linker or an albumin binding domain; X² is a linker or an albumin binding domain; Z¹ is a nuclear localization domain; and Z² is a nuclear localization domain.

In some embodiments, X¹ is repeated three times and at each occurrence is independently a linker or an albumin binding domain; and Z¹ is a nuclear localization domain.

In some embodiments, X² is repeated three times and at each occurrence is independently a linker or an albumin binding domain; and Z² is a nuclear localization domain.

In some embodiments, X¹ is repeated three times and at each occurrence is independently a linker or an albumin binding domain; X² is not present; Z¹ is a nuclear localization domain; and Z² is a nuclear localization domain.

In some embodiments, X¹ is repeated three times and at each occurrence is independently a linker or an albumin binding domain; X² is repeated three times and at each occurrence is independently a linker or an albumin binding domain; Z¹ is a nuclear localization domain; and Z² is a nuclear localization domain.

The recombinant protein can further include other domains that can aid in the pharmacokinetics, tissue localization, cellular localization, or a combination thereof of the recombinant protein. In addition, the recombinant protein can further include additional amino acid sequences at the C-terminal end and/or N-terminal end. These amino acid sequences may aid in purification, expression, stability, or another desired property. Example amino acid sequences can include, but are not limited to, an HRV3 C protease cleavage site, a CL7 tag, a polyhistidine tag, and a linker as described herein. Combinations of the foregoing amino acid sequences can be used.

The recombinant protein can have a varying molecular weight. For example, the recombinant protein can have a molecular weight of about 100 kilodaltons (kDa) to about 400 kDa, such as about 110 kDa to about 350 kDa, about 120 kDa to about 320 kDa, about 100 kDa to about 300 kDa, about 100 kDa to about 200 kDa, about 110 kDa to about 220 kDa, about 130 kDa to about 210 kDa, or about 160 kDa to about 190 kDa. In some embodiments, the recombinant protein has a molecular weight of at least 100 kDa, at least 120 kDa, at least 140 kDa, or at least 160 kDa. In some embodiments, the recombinant protein has a molecular weight of no more than 400 kDa, no more than 300 kDa, no more than 250 kDa, or no more than 200 kDa. Molecular weight of the recombinant protein can be measured by techniques known within the art such as, but not limited to, matrix assisted laser desorption/ionization (MALDI) mass spectrometry.

The recombinant protein can advantageously bind to albumin through the albumin binding domain(s). For example, the recombinant protein can have a binding affinity (K_{D}) to albumin of less than 1 μM, less than 500 nM, less than 250 nM, less than 100 nM, less than 80 nM, less than 60 nM, less than 50 nM, less than 45 nM, less than 40 nM, less than 35 nM, less than 30 nM, less than 25 nM, less than 20 nM, or less than 15 nM. In some embodiments, the recombinant protein has a Kp to albumin of greater than 0.1 nM, greater than 0.2 nM, greater than 0.4 nM, greater than 0.5 nM, greater than 0.6 nM, greater than 0.7 nM, greater than 0.8 nM, greater than 0.9 nM, greater than 1 nM, greater than 5 nM, or greater than 10 nM. In some embodiments, the recombinant protein has a Ko to albumin of about 0.1 nM to about 1 μM, such as about 0.5 nM to about 500 nM, about 0.8 nM to about 100 nM, about 0.5 nM to about 100 nM, about 1 nM to about 50 nM, about 5 nM to about 100 nM, about 10 nM to about 60 nM, or about 5 nM to about 75 nM. The recombinant protein can reversibly bind albumin. In some embodiments, the recombinant protein does not covalently bind to albumin. Binding affinity can be measured by techniques known within the art such as, but not limited to, competitive binding assays and biolayer interferometry.

### A. Cas9 Domain

The Cas9 domain can include a Cas9 nuclease, such as a spCas9 nuclease. In some embodiments, the Cas9 domain is part of a ribonucleoprotein. The Cas9 domain can interact with one or more gRNA as discussed herein, e.g., by forming a complex with the 3′ end of the gRNA. In concert with the gRNA(s), the recombinant protein can localize to a site which includes a target domain of a target DNA. In some embodiments, the Cas9 domain includes an amino acid sequence of SEQ ID NO:29. In some embodiments, the Cas9 domain consists of an amino acid sequence of SEQ ID NO:29.

### B. Albumin Binding Domain

The recombinant protein can include an albumin binding domain. The albumin binding domain can instill in the recombinant protein the ability to bind albumin, e.g., upon systemic administration. This albumin hitchhiking approach can extend circulation half-life and inflamed tissue accumulation of the recombinant proteins, while also decreasing immunogenicity and/or recognition by pre-existing immunity. The albumin binding domain can include a peptide that is capable of specifically binding to albumin. The recombinant protein may include more than one albumin binding domain. For example, the recombinant protein can include 1 albumin binding domain, 2 albumin binding domains, 3 albumin binding domains, 4 albumin binding domains, 5 albumin binding domains, or more.

In some embodiments, the recombinant protein includes 1 to 4 albumin binding domains, such as 1 to 2, 1 to 3, or 2 to 3. In some embodiments, the recombinant protein includes at least 1 albumin binding domain, at least 2 albumin binding domains, or at least 3 albumin binding domains. In some embodiments, the recombinant protein includes no more than 4 albumin binding domains, no more than 3 albumin binding domains, or no more than 2 albumin binding domains.

In some embodiments, the albumin binding domain includes an amino acid sequence selected from the group consisting of SEQ ID NO:20, SEQ ID NO:30, SEQ ID NO:31, SEQ ID NO: 32, SEQ ID NO:33, SEQ ID NO:34, SEQ ID NO:35, and a combination thereof. In some embodiments, the albumin binding domain includes an amino acid sequence selected from the group consisting of SEQ ID NO:20, SEQ ID NO:30, SEQ ID NO:31, SEQ ID NO:32, SEQ ID NO: 33, SEQ ID NO:34, and SEQ ID NO:35.

In some embodiments, the albumin binding domain includes an amino acid sequence of SEQ ID NO:20. In some embodiments, the albumin binding domain consists of an amino acid sequence of SEQ ID NO:20.

### C. Nuclear Localization Domain

The recombinant protein can also include a nuclear localization domain. The nuclear localization domain can instill in the recombinant protein improved cellular uptake, nuclear localization, or both. The nuclear localization domain can include SV40. The recombinant protein may include more than one nuclear localization domain. For example, the recombinant protein can include 1 nuclear localization domain, 2 nuclear localization domains, 3 nuclear localization domains, 4 nuclear localization domains, 5 nuclear localization domains, or more.

In some embodiments, the recombinant protein includes 1 to 10 nuclear localization domains, such as 1 to 3, 1 to 6, 2 to 6, or 3 to 6. In some embodiments, the recombinant protein includes at least 1 nuclear localization domain, at least 2 nuclear localization domains, at least 3 nuclear localization domains, at least 4 nuclear localization domains, or at least 5 nuclear localization domains. In some embodiments, the recombinant protein includes no more than 6 nuclear localization domains, no more than 5 nuclear localization domains, no more than 4 nuclear localization domains, no more than 3 nuclear localization domains, or no more than 2 nuclear localization domains.

In some embodiments, the nuclear localization domain includes an amino acid sequence selected from the group consisting of SEQ ID NO:21, SEQ ID NO:36-SEQ ID NO: 46, and a combination thereof. In some embodiments, the nuclear localization domain includes an amino acid sequence selected from the group consisting of SEQ ID NO:21 and SEQ ID NO:36-SEQ ID NO:46.

In some embodiments, the nuclear localization domain includes an amino acid sequence of SEQ ID NO:21. In some embodiments, the nuclear localization domain includes an amino acid sequence of SEQ ID NO:21 repeated 1 to 3 times. In some embodiments, the nuclear localization domain includes an amino acid sequence of SEQ ID NO:21 repeated 3 times. In some embodiments, the nuclear localization domain includes an amino acid sequence of SEQ ID NO:21 repeated 3 times with aspartic acid (D) between repeats. In some embodiments, the nuclear localization domain includes an amino acid sequence of SEQ ID NO: 38.

### D. Linker

The recombinant protein can include a linker. The linker can be placed at specified locations within the recombinant protein, such as between different domains. The linker can provide structural flexibility or rigidity to the different domains. For example, a linker may be included in the recombinant protein at locations to allow the albumin binding domain steric flexibility to bind albumin. In some embodiments, the recombinant protein includes a linker between the albumin domain and the nuclear localization domain. In some embodiments, the recombinant protein includes a linker between the albumin binding domain and the Cas9 domain. In some embodiments, the recombinant protein includes a linker between the Cas9 domain and the nuclear localization domain.

The recombinant protein can include a varying number of linkers depending on the other domains present in the protein. For example, the recombinant protein can include at least 1 linker, at least 2 linkers, at least 3 linkers, at least 4 linkers, or at least 5 linkers. In some embodiments, the recombinant protein includes no more than 5 linkers, no more than 4 linkers, no more than 3 linkers, or no more than 2 linkers. In some embodiments, the recombinant protein includes 1 to 10 linkers, such as 1 to 8, 1 to 5, 2 to 6, or 1 to 3.

The linker can be a peptide linker. In some embodiments, the linker includes an amino acid sequence selected from the group consisting of SEQ ID NO:22, SEQ ID NO:47-SEQ ID NO:57, and a combination thereof. In some embodiments, the linker includes an amino acid sequence selected from the group consisting of SEQ ID NO:22 and SEQ ID NO:47-SEQ ID NO: 57. In some embodiments, the linker includes an amino acid sequence selected from the group consisting of SEQ ID NO:22, SEQ ID NO:47-SEQ ID NO:51, and a combination thereof. In some embodiments, the linker includes an amino acid sequence selected from the group consisting of SEQ ID NO:22 and SEQ ID NO:47-SEQ ID NO:51.

The linker can be a glycine linker, e.g., including 1 to 30 glycine amino acids, such as 1 to 20, 1 to 15, or 1 to 10. In some embodiments, the linker includes an amino acid sequence selected from the group consisting of SEQ ID NO:22, SEQ ID NO:47, SEQ ID NO:48, and a combination thereof. In some embodiments, the linker includes an amino acid sequence selected from the group consisting of SEQ ID NO:22, SEQ ID NO:47, and SEQ ID NO:48. In some embodiments, the linker includes an amino acid sequence of SEQ ID NO:22. In some embodiments, the linker consists of an amino acid sequence of SEQ ID NO:22.

### E. Example Recombinant Proteins

In some embodiments, the recombinant protein includes an amino acid sequence that is at least 95% identical to any one of SEQ ID NO:24, SEQ ID NO:25, SEQ ID NO:26, SEQ ID NO: 27, and SEQ ID NO:28.

In some embodiments, the recombinant protein includes an amino acid sequence that is at least 99% identical to any one of SEQ ID NO:24, SEQ ID NO:25, SEQ ID NO:26, SEQ ID NO: 27, and SEQ ID NO:28.

In some embodiments, the recombinant protein includes an amino acid sequence of any one of SEQ ID NO:24, SEQ ID NO:25, SEQ ID NO:26, SEQ ID NO:27, and SEQ ID NO:28.

In some embodiments, the recombinant protein includes an amino acid sequence that is at least 95% identical to any one of SEQ ID NO:26 and SEQ ID NO:27.

In some embodiments, the recombinant protein includes an amino acid sequence that is at least 99% identical to any one of SEQ ID NO:26 and SEQ ID NO:27.

In some embodiments, the recombinant protein includes an amino acid sequence of any one of SEQ ID NO:26 and SEQ ID NO:27.

In some embodiments, the recombinant protein includes an amino acid sequence that is at least 95% identical to SEQ ID NO:26.

In some embodiments, the recombinant protein includes an amino acid sequence that is at least 99% identical to SEQ ID NO:26.

In some embodiments, the recombinant protein includes an amino acid sequence of SEQ ID NO:26.

### 3. Guide RNAs

In another aspect, the present disclosure further provides a gRNA molecule (e.g., an isolated or non-naturally occurring RNA molecule) that can interact with a Cas protein. In certain embodiments, the gRNA is an sgRNA, in which the crRNA (i.e., the targeting domain or complementary region) targets a human gene. In certain embodiments, the targeting domain is a crRNA that is provided to a eukaryotic cell with tracrRNA, which acts as a scaffold through interactions with both the crRNA and a Cas protein. In some embodiments, the system is further, optionally, comprised of an oligonucleotide—an HDR template with homology to either side of the target position (see Bloh, K., & Rivera-Torres, N, at 3836, which is incorporated by reference herein in its entirety).

In some embodiments, the crRNA of the gRNA molecule is configured to orient an associated nuclease such that a cleavage or gene regulation event occurs (e.g., a double strand break or a locus), thereby facilitating an alteration in the nucleic acid sequence. In some embodiments, the crRNA is 20 nucleotides in length. In some embodiments, the crRNA is 14, 15, 16, 17, 18, 19, 20, 21, 22, 23, 24, 25, 26, 27, 28, 29, or 30 nucleotides in length. In some embodiments, the crRNA orients the nuclease such that a cleavage event occurs within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of a target position. The double-strand or single-strand break may be positioned upstream or downstream of a target position, and either within or upstream a functional domain cluster within the targeted gene.

In certain embodiments, a second gRNA molecule, comprising a second crRNA orients a second associated nuclease such that a cleavage event occurs sufficiently close to a target position, in the targeted gene or locus, thereby facilitating an alteration in the nucleic acid sequence. In some embodiments, the second gRNA molecule targets the same targeted gene or locus as the first gRNA molecule. In other embodiments, the second gRNA molecule targets a different targeted gene or locus as the first gRNA molecule. In some embodiments, the second crRNA is 20 nucleotides in length. In some embodiments, the second crRNA is 14, 15, 16, 17, 18, 19, 20, 21, 22, 23, 24, 25, 26, 27, 28, 29, or 30 nucleotides in length.

In some embodiments, the second crRNA orients the nuclease such that a cleavage event occurs within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of a target position. The double-strand or single-strand break, may be positioned upstream or downstream of a target position, and either within or upstream a functional domain cluster within the targeted gene.

In some embodiments, the crRNAs of a first and second gRNA molecules are configured such that a cleavage event is positioned, independently for each of the gRNA molecules, within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of the respective target position. In certain embodiments, the first and second gRNA molecules alter the targeted nucleic acid sequences simultaneously. In certain embodiments, the first and second gRNA molecules alter the targeted nucleic acid sequences sequentially, strand break, positioned by the crRNAs of a first and second gRNA molecule, respectively. For example, the crRNAs may orient the associated nucleases such that a cleavage event, (e.g., the two single-strand breaks), are positioned within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of a target position. In some embodiments, the crRNA of a first and second gRNA molecules are configured to orient associated nucleases such that, for example, two single-strand breaks occur at the same target position, or within 1, 2, 3, 4, 5, 10, 15, 20, 25, 30, 35, 40, 45, or 50 nucleotides of one another, on opposing strands of genomic DNA, thereby essentially approximating a double strand break.

The recombinant protein of the present disclosure can be used with at least one gRNA molecule. In some embodiments, multiple gRNA molecules (e.g., two gRNA molecules) are combined with a single Cas9 protein, such that the gRNA provides the targeting of a Cas9 nuclease. In some embodiments, the gRNA molecule is a sgRNA molecule. The sgRNA is a fusion of two noncoding RNAs: a crRNA and a tracrRNA. The sgRNA may target any desired DNA sequence by exchanging the sequence encoding a 20 bp protospacer which confers targeting specificity through complementary base pairing with the desired DNA target. The gRNA can mimic the naturally occurring crRNA: tracrRNA duplex involved in the Type II Effector system. This duplex, which may include, for example, a 42-nucleotide crRNA and a 75-nucleotide tracrRNA, can act as a guide for Cas9 to cleave a targeted polynucleotide. In some embodiments, containing two or more gRNA molecules, each gRNA targets a different DNA sequence. The target DNA sequences may be overlapping. The target sequence or protospacer can be followed by a PAM sequence at the 3′ end of the protospacer. Different Type II systems have differing PAM requirements. For example, the Streptococcus pyogenes Type II system uses an “NGG” sequence, where “N” can be any nucleotide. In some embodiments, the PAM sequence may be “NGG,” where “N” can be any nucleotide.

The gRNA molecule can comprise a targeting domain (also referred to as a targeting sequence or cRNA sequence), which is a complementary polynucleotide sequence of the target DNA sequence followed by a PAM sequence. The gRNA may comprise a “G” at the 5′ end of the targeting domain or complementary polynucleotide sequence. The targeting domain of a gRNA molecule may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. The targeting domain of a gRNA molecule may comprise less than a 40 base pair, less than a 35 base pair, less than a 30 base pair, less than a 25 base pair, less than a 20 base pair, less than a 19 base pair, less than a 18 base pair, less than a 17 base pair, less than a 16 base pair, less than a 15 base pair, less than a 14 base pair, less than a 13 base pair, less than a 12 base pair, less than a 11 base pair, or less than a 10 base pair complementary polynucleotide sequence of the target DNA sequence followed by a PAM sequence. In certain embodiments, the targeting domain of a gRNA molecule is 19-25 nucleotides in length. In certain embodiments, the targeting domain of a gRNA molecule is 20 nucleotides in length. In certain embodiments, the targeting domain of a gRNA molecule is 21 nucleotides in length. In certain embodiments, the targeting domain of a gRNA molecule is 22 nucleotides in length. In certain embodiments, the targeting domain of a gRNA molecule is 23 nucleotides in length.

The gRNA molecules of the present disclosure may target various mutant genes in a subject to treat a genetic disease associated with said gene(s). In some embodiments, the gRNA targets any one of the genes selected from the dystrophin gene (i.e., the DMD gene; NCBI Gene ID: 1756), a neurotrophin gene (e.g., the NT3 gene; NCBI Gene ID: 4908), the huntingtin gene (i.e., the HTT gene; NCBI Gene ID: 3064), a calpain gene (e.g., the CAPN3 gene; NCBI Gene ID: 825), the dysferlin gene (i.e., the DYSF gene; NCBI Gene ID: 8291), the alpha-sarcoglycan gene (i.e., the SGCA gene; NCBI Gene ID: 6442), the beta-sarcoglycan gene (i.e., the SGCB gene; NCBI Gene ID: 6443), the delta-sarcoglycan gene (i.e., the SGCD gene; NCBI Gene ID: 6444), the gamma-sarcoglycan gene (i.e., the SGCG gene; NCBI Gene ID: 6445), the telethonin gene (i.e., the TCAP gene; NCBI Gene ID: 8556), the fukutin-related protein gene (i.e., the FKRP gene; NCBI Gene ID: 79147), the anoctamin 5 gene (i.e., the ANO5 gene: NCBI Gene ID: 203859), an ataxin gene (e.g., the ATX1 gene; NCBI Gene ID: 6310), the DM1 gene (i.e., the DMPK gene; NCBI Gene ID: 1760), and the DM2 gene (i.e., the CNBP gene; NCBI Gene ID: 7555).

### 4. Genetic Constructs

In another aspect, provided are genetic constructs encoding the recombinant protein and/or gRNA detailed herein. A vector may include a polynucleotide encoding the recombinant protein and/or gRNA detailed herein. The genetic construct, such as a plasmid or expression vector, may comprise a nucleic acid that encodes the recombinant protein and/or the gRNA.

Genetic constructs may include polynucleotides such as vectors and plasmids. The genetic construct may be a linear minichromosome including centromere, telomeres, or plasmids or cosmids. The vector may be an expression vectors or system to produce protein by routine techniques and readily available starting materials including Sambrook et al., Molecular Cloning and Laboratory Manual, Second Ed., Cold Spring Harbor (1989), which is incorporated fully herein by reference. The construct may be recombinant. The genetic construct may be part of a genome of a recombinant viral vector, including recombinant lentivirus, recombinant adenovirus, and recombinant adenovirus associated virus. The genetic construct may comprise regulatory elements for gene expression of the coding sequences of the nucleic acid. The regulatory elements may be a promoter, an enhancer, an initiation codon, a stop codon, or a polyadenylation signal.

The genetic construct may include heterologous nucleic acid encoding the recombinant protein and/or gRNA detailed herein and may further include an initiation codon, which may be upstream of the recombinant protein and/or gRNA coding sequence, and a stop codon, which may be downstream of the recombinant protein and/or gRNA coding sequence. The genetic construct may include more than one stop codon, which may be downstream of the recombinant protein and/or gRNA coding sequence. In some embodiments, the genetic construct includes 1, 2, 3, 4, or 5 stop codons.

The vector may also include a promoter that is operably linked to the recombinant protein and/or gRNA coding sequence. The promoter may be a constitutive promoter, an inducible promoter, a repressible promoter, or a regulatable promoter. The promoter may be a ubiquitous promoter. The promoter may be a tissue-specific promoter. The tissue specific promoter may be a muscle specific promoter. A promoter operably linked to the recombinant protein and/or gRNA coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter.

The genetic construct may also comprise a polyadenylation signal, which may be downstream of the recombinant protein and/or gRNA coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 vector (Invitrogen, San Diego, CA).

The genetic construct may also comprise an enhancer upstream of the recombinant protein and/or gRNA coding sequence. The enhancer may be necessary for DNA expression. The enhancer may be human actin, human myosin, human hemoglobin, human muscle creatine or a viral enhancer such as one from CMV, HA, RSV, or EBV. Polynucleotide function enhancers are described in U.S. Pat. Nos. 5,593,972, 5,962,428, and WO94/016737, the contents of each are fully incorporated by reference herein. The genetic construct may also comprise a mammalian origin of replication in order to maintain the vector extrachromosomally and produce multiple copies of the vector in a cell. The genetic construct may also comprise a regulatory sequence, which may be well suited for gene expression in a mammalian or human cell into which the vector is administered. The genetic construct may also comprise a reporter gene, such as green fluorescent protein (“GFP”) and/or a selectable marker, such as hygromycin (“Hygro”).

### 5. Compositions

In another aspect, disclosed are compositions that include the recombinant protein and one or more gRNAs. In some embodiments, the composition includes 1 or 2 gRNAs. In some embodiments, the gRNA is a single-guide RNA (sgRNA) that includes a crRNA attached to a tracrRNA. In some embodiments, the composition is a “DNA Targeting System,” which refers to a system capable of specifically targeting a particular region of DNA and modulating gene expression by binding to that region. An example of these systems is a CRISPR/Cas-based gene editing system, in which the Cas domain of the recombinant protein with at least one gRNA targeting the Cas domain to a target region of the target DNA.

The compositions can also include one or more pharmaceutically acceptable excipients, where such compositions can also be referred to as a pharmaceutical composition. Examples of pharmaceutically acceptable excipients include, but are not limited to, buffering agents (e.g., phosphate buffered saline), carbohydrates (e.g., glucose, trehalose, starch, etc.) solubilizers, solvents, antimicrobial preservatives, antioxidants, suspension agents, or a combination thereof.

In another aspect, disclosed are compositions for use in the treatment of a genetic disease, the composition including a recombinant protein and one or more gRNAs as disclosed herein. Example genetic diseases include, but are not limited to, Duchenne muscular dystrophy (DMD), Limb Girdle muscular dystrophy (LGMD), myotonic dystrophy, Charcot-Marie Tooth Syndrome (CMT), Spinal Cerebellar Ataxia, and Huntington's disease.

The description of the recombinant protein, nuclear localization domain, albumin binding domain, Cas9 domain, linker, genetic construct, and gRNA may be applied to the disclosed compositions.

### 6. Uses of the Recombinant Proteins

As discussed elsewhere herein, the recombinant protein can bind albumin through the albumin binding domain. In some embodiments, the binding of the recombinant protein to albumin (e.g., serum albumin) enhances the pharmacokinetic properties of the recombinant protein as compared to the recombinant protein lacking the albumin binding domain. In some embodiments, enhancing the pharmacokinetic properties includes increasing the circulation half-life and/or bioavailability of the disclosed recombinant protein, as compared to the recombinant protein lacking the albumin binding domain. Additionally, enhancing the pharmacokinetic properties may include increasing the quantity of cellular accumulation, increasing the homogeneity of cellular or tissue accumulation, increasing resistance to nucleases, and/or permitting increased dosing amount with decreased toxicity as compared to the recombinant protein lacking the albumin binding domain. The ability to bind to albumin can be beneficial in methods as disclosed below.

### A. Methods of Modulating Gene Expression

In another aspect, disclosed herein are methods of modulating gene expression in a subject (e.g., in need thereof). The method can include administering to the subject a therapeutically effective amount of the recombinant protein, gRNA, or composition thereof.

The expression of the gene may be modulated by at least 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be modulated by less than 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be modulated by about 5% to about 95%, about 10% to about 90%, about 15% to about 85%, about 20% to about 80%, or about 1.5-fold to about 10-fold, relative to a control.

Modulating may include, e.g., increasing or enhancing expression of the gene. The expression of the gene may be increased by at least 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be increased by less than 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be increased by about 5% to about 95%, about 10% to about 90%, to about 15% to about 85%, about 20% to about 80%, or about 1.5-fold to about 10-fold, relative to a control.

Modulating may also include, e.g., reducing or inhibiting expression of the gene. The expression of the gene may be reduced by at least 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be reduced by less than 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be reduced by about 5% to about 95%, about 10% to about 90%, about 15% to about 85%, about 20% to about 80%, or about 1.5-fold to about 10-fold, relative to a control.

### B. Methods of Treating a Genetic Disease

In another aspect, disclosed herein are methods of modulating gene expression in a subject or patient (e.g., in need thereof). The method can include administering to the patient a therapeutically effective amount of the recombinant protein, gRNA, or composition thereof. In some embodiments, the method includes administering a therapeutically effective amount of a composition including the recombinant protein and one or more gRNAs. The method can be used to treat a number of genetic disease including, but not limited to, Duchenne muscular dystrophy (DMD), Limb Girdle muscular dystrophy (LGMD), myotonic dystrophy, Charcot-Marie Tooth Syndrome (CMT), Spinal Cerebellar Ataxia, and Huntington's disease.

### C. Methods of Modifying Targeted Genes

In another aspect, disclosed herein are methods of modifying a targeted gene associated with disease in a subject or a patient (e.g., in need thereof). The method can include administering to the patient a therapeutically effective amount of the recombinant protein, gRNA, or composition thereof. In some embodiments, the method includes administering a therapeutically effective amount of a composition including the recombinant protein and one or more gRNAs. In some embodiments, the targeted gene is selected from any one of a dystrophin (DMD) gene, a calpain 3 (CAPN3) gene, a dysferlin (DYSF) gene, an alpha-sarcoglycan (SGCA) gene, a beta-sarcoglycan (SGCB) gene, a gamma-sarcoglycan (SGCG) gene, a delta-sarcoglycan gene (SGCD) gene, a telethonin (TCAP) gene, a fukutin-related protein (FKRP) gene, an anoctamin 5 (ANO5) gene, a DM1 gene, a DM2 gene, a neurotrophin 3 (NT3) gene, and a huntingtin (HTT) gene. In some embodiments, the targeted gene is a human gene.

### D. Administration

The recombinant protein, gRNA, or composition thereof can be administered prophylactically or therapeutically. In prophylactic administration, the recombinant protein, gRNA, or composition thereof can be administered in an amount sufficient to induce a response. In therapeutic applications, the recombinant protein, gRNA, or composition thereof can be administered to a subject in need thereof in an amount sufficient to elicit a therapeutic effect. Amounts effective for this use will depend on, e.g., the particular regimen administered, the manner of administration, the stage and severity of the disease, the general state of health of the patient, and the judgment of the prescribing physician.

The recombinant protein, gRNA, or composition thereof as detailed herein may be administered or delivered to a cell, to a subject, or to a cell in a subject. The recombinant protein and the gRNA can be administered separately or together. The recombinant protein, the gRNA, or composition thereof may be administered in dosages and by techniques well known to those skilled in the medical arts taking into consideration such factors as the age, sex, weight, and condition of the particular subject, and the route of administration.

The recombinant protein, gRNA, or composition thereof may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular, or combinations thereof. The recombinant protein, gRNA, or composition thereof may be delivered to a subject by several technologies including stereotactic injection, robotic implantation (e.g., Neuralink, Neuralink Corporation, San Francisco, CA), and the like.

The nucleic acids and/or genetic constructs as described herein, or compositions including the same, may be delivered to a subject by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. Any of the nucleic acids and/or genetic constructs may be injected into the skeletal muscle or cardiac muscle. The nucleic acids and/or genetic constructs or compositions including the same may be administered by traditional syringes, needleless injection devices, “microprojectile bombardment gone guns,” or other physical methods such as electroporation (“EP”), “hydrodynamic method”, intravenous injection, retro-orbital injection, or ultrasound.

As will be readily apparent to one skilled in the art, the useful in vivo dosage to be administered and the particular mode of administration will vary depending upon the age, weight, the severity of the affliction, and subjects treated, and the specific use. The determination of effective dosage levels, that is the dosage levels necessary to achieve the desired result, can be accomplished by one skilled in the art using routine methods, for example, human clinical trials, in vivo studies and in vitro studies.

Dosage amount and interval may be adjusted individually to provide, e.g., plasma levels of the biologically active agent which are sufficient to maintain the modulating effects, or minimal effective concentration (MEC). The MEC will vary for each agent but can be estimated from in vivo and/or in vitro data. Dosages necessary to achieve the MEC will depend on individual characteristics and route of administration. However, assays well known to those in the art can be used to determine plasma concentrations. Dosage intervals can also be determined using MEC value. Compositions can be administered using a regimen which maintains plasma levels above the MEC for 10-90% of the time, such as 30-90% or 50-90%. In cases of local administration or selective uptake, the effective local concentration of the drug may or may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response was not adequate (precluding toxicity). The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be used in veterinary medicine.

The description of the recombinant protein, nuclear localization domain, albumin binding domain, Cas9 domain, linker, genetic construct, gRNA, and compositions may be applied to the disclosed methods and uses.

### 7. Kits

In another aspect, disclosed herein are kits. The kit may include the recombinant protein, the gRNA, compositions thereof, or a genetic construct encoding the recombinant protein and/or the gRNA. In some embodiments, the kit includes a DNA targeting system or a CRISPR/Cas-based gene editing system.

The kit can include instructions. Instructions may be affixed to packaging material or may be included as a package insert. While the instructions are typically written on printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The description of the recombinant protein, nuclear localization domain, albumin binding domain, Cas9 domain, linker, genetic construct, gRNA, and compositions may be applied to the disclosed kits.

### 8. Examples

The disclosed technology has multiple aspects, illustrated by the following non-limiting examples.

### Example 1

### Materials & Methods

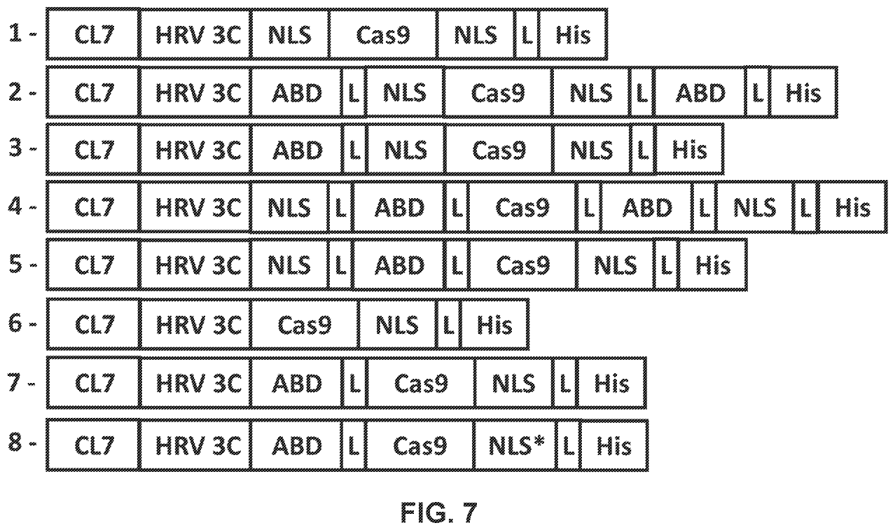

Recombinant Cas9 Protein Production. S. pyogenes Cas9 (SpCas9) was used. First, plasmids were transformed into E. coli Tuner DE3 pLacl competent cells (Sigma) from plasmids based on pCold CL7-Cas9 (Addgene plasmid #124890) according to standard heat-shock protocol. All constructs had an N-terminal CL7 tag (CL7 is a catalytically inactive variant of Colicin E7 (CE7) DNase) and a C-terminal 6× histidine (HIS) tag. NLS from the SV40 large T-antigen, albumin binding domains (ABD), and HIS tag modifications were cloned into the plasmid using the Gibson DNA assembly method (NEB) according to manufacturing instructions. All constructs were verified by direct sanger sequencing after cloning. Recombinant plasmids were then grown in terrific broth (TB) with 100 ug/ml ampicillin at 18° C. for 16-18 hours following growth induction with 0.5 mM IPTG. Bacterial cells were then lysed with a sonicator at 0.5 s on/off repeated three times. Cells were then centrifuged at 14,000 rpm for 1 hour. Cas9 protein in soluble fraction was then purified by sequential Ni²⁺-nitriloacetic acid column (QIAGEN), Heparin (Cytiva HiTrap), and Lm7 columns (TriAltus Bioscience) purifications according to manufacturer instructions. Eluted samples were passed through MW cutoff and buffer exchanged into “Injectable Freezing Buffer” or IFB and stored at −80° C. Cleaved CL7 tag and Prescission Protease were efficiently removed by MW cutoff. Proteins were then quantified using BSA assay. Finally, Cas9 protein endotoxin levels were measured using Pierce LAL Chromogenic Endotoxin Quantification Kit Cat. #88282. Purity of recombinant proteins was determined by running a 4-20% SDS gel and staining with Coomassie blue. Sequences used for cloning are shown below. Sequence architecture is shown in FIG. 7.

Cas9 RNP Formation. Cas9 RNP complexes were either made immediately prior to experiments or prepared and stored at −80° C. for later use. Before RNP formation, guide RNAs were re-annealed on a thermocycler by heating to 85° C. and then cooling to 25° C. over 4 minutes. Cas9 Protein was mixed with guide RNA at a 1:1.25 molar ratio and incubated at 37° C. for 30 minutes. Excess gRNA was removed from the RNPs and buffer was exchanged by two 10× dilution and re-concentration steps using 100 kDa MWCO spin columns for a 100-fold total dilution of the original solution. Protein concentration was verified by both A280 and BCA Assay. RNP formation and excess-RNA removal was confirmed by verification of A260/A280 ratios.

In vitro cleavage assay. An Ai9 plasmid (Addgene #22799) was used for all in vitro cleavage assays. dsDNA was linearized with Xhol (NEB) according to manufacturer protocol. Linearized fragments were then analyzed with gel electrophoresis and purified using Gel extraction kit (Qiagen) according to manufacturer instructions. Cleavage assays were conducted in a reaction volume of 20 μl with RNPs containing 20 nM SpCas9, 28 nM sgRNA, and 2 nM linearized Ai9 dsDNA substrate in dPBS with 100 nM NaCl. For albumin binding studies, either 20 nM or 100 nM human serum albumin (HSA) was added to the reaction. Reactions were incubated at 37° C. for 1 hour followed by addition of 1 ug/ul of Proteinase K with incubation at 56° C. for 15 minutes. Cleaved dsDNA fragments were run through gel electrophoresis in 0.7% agarose gel and percent editing was measured with densitometry using BioRad Image Lab software.

Biolayer Interferometry. Binding kinetics of the different Cas9 recombinant proteins with albumin was measured by biolayer interferometry (BLI) using the Octet R38 system (FortéBio). Streptavidin (SA) dip and read biosensors (Sartorius) were used for this study and were rehydrated in ddH20 for 10 minutes prior to kinetics experiment. Assay protocol was run as follows: SA biosensors were washed with dPBS for 180 s to establish baseline. Then, 25 ug/ml biotinylated human serum albumin (HSA) was loaded for 300 s followed by washing with dPBS for 60 s. Association to recombinant Cas9 proteins with or without ABD was measured for 400 seconds at different concentrations ranging from 1 μM to 0 uM. Dissociation was then measured by loading tips into dPBS for 400 seconds. All steps were performed at 30° C. with an agitation speed of 1000 rpm. Data was analyzed using Octet Data Analysis HT Software. Interstep correction was performed by aligning to the association step. Global analysis and 1:1 binding kinetics was performed to derive Ko's simultaneously from all tested analyte concentrations.

Animal work. All mouse procedures were reviewed and approved by Vanderbilt University's IACUC. Housing was standard (12 h light-dark cycle, 40-60% humidity, 18-23° C.). For systemic editing studies, Ai9 mice (Jackson Laboratory RRID: IMSR JAX: 007909) were used. To study biodistribution, CD-1 (Charles River) mice were used. Mice were utilized of both sexes and similar age and weight (18-20 g; 6-8 weeks old). All tail vein procedures were performed using a mouse tail illuminator restrainer to both restrain and warm tail before injections.

Muscle inflammation model. Barium chloride injection was used to emulate the phenotype of inflamed muscle tissue. Mice were first anesthetized with isoflurane (1.5-3%), fur shaved over muscle area, followed by 40 μL intramuscular injection with 1.2% barium chloride in 0.9% sterile saline in tibialis anterior muscle. The contralateral muscle was injected with 40 μL of 0.9% sterile saline unless otherwise noted. Forty-eight hours after injection, pharmacokinetics, biodistribution, and gene-editing studies were conducted as described below.

Blood plasma pharmacokinetics and biodistribution. SpCas9 RNPs with fluorescently (Cy-5-labeled) protein were injected (i.v.) into the tail vein of CD-1 mice (4 to 6-wk-old, Charles Rivers Laboratories) at 0.5 mg/kg. Immediately following injection, blood was serially sampled at 5, 30, 60, and 120 minutes in EDTA-coated tubes and Cy5 fluorescence measured in 96-well plates by fluorimetry (Tecan). Following the 120-minute time-mark, whole organs (heart, lungs, liver, kidneys, spleen, and skeletal muscle) were harvested and Cy5 fluorescence was quantified in whole organs using IVIS Lumina Imaging system (Xenogen Corporation).

In vivo Intramuscular Administration. Ai9 mice were intramuscularly injected with 0.1 nmol (~16 μg) spCas9 RNP into the tibialis anterior muscle. One week later, mice were sacrificed, legs were harvested, and tdTomato turn-on was assessed by IVIS, cryohistology, and IDAA as described in their respective methods sections.

In vivo Intravenous Administration for gene-editing. 48 hours after barium chloride injury, Ai9 mice were intravenously injected with 2 nmol spCas9 RNP (2 mg/mL, ~150 UL, ~15 mg/kg. Two-weeks later, mice were sacrificed, organs (heart, lung, liver, spleen, kidneys, legs, and tibialis anterior muscle) harvested, and tdTomato turn-on was assessed by IVIS, cryohistology, and IDAA as described in their respective methods sections.

Cryohistology. Following IVIS, mouse hindlimbs were dissected and fixed in 2% PFA for 3 hours at 4° C. and immersed in 15% sucrose followed by 30% sucrose overnight at 4° C. Following the sucrose gradient, the tibialis anterior was excised and embedded into O.C.T compound and immediately frozen in liquid nitrogen. Cryosectioning was performed using a Leica CM1950 Cryostat at a thickness of 7-8 μm. Slides were mounted with ProLong Gold Antifade Mountant containing DAPI for nuclear visualization. The entire area with tissue on each slide was then imaged using the Nikon TI-Eclipse/C1 Confocal.

IVIS Imaging. IVIS images were acquired on an IVIS Lumina Imaging System (Xenogen Corporation). Spectral Unmixing settings were used, with total excitation emission combinations taken. In the Living Image software, the guided unmixing steps were followed to detect tdTomato expression in the respective tissue, and the same settings were applied to each tissue throughout this work using the Library option.

### Example 2

### Characterization of Example Recombinant Cas9 Proteins

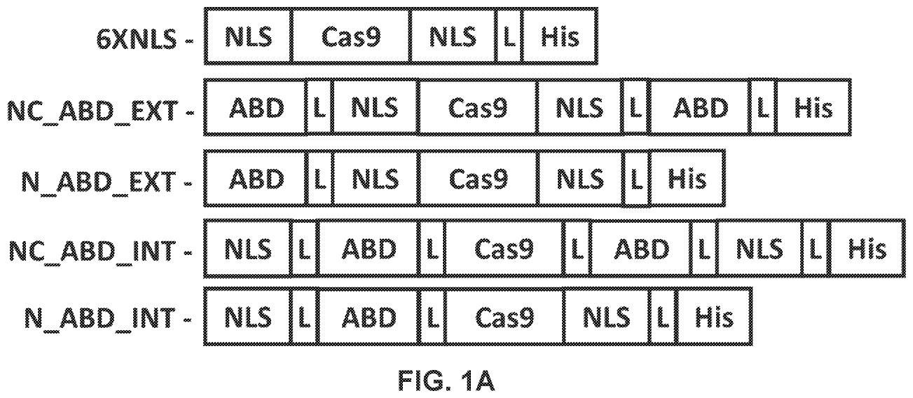

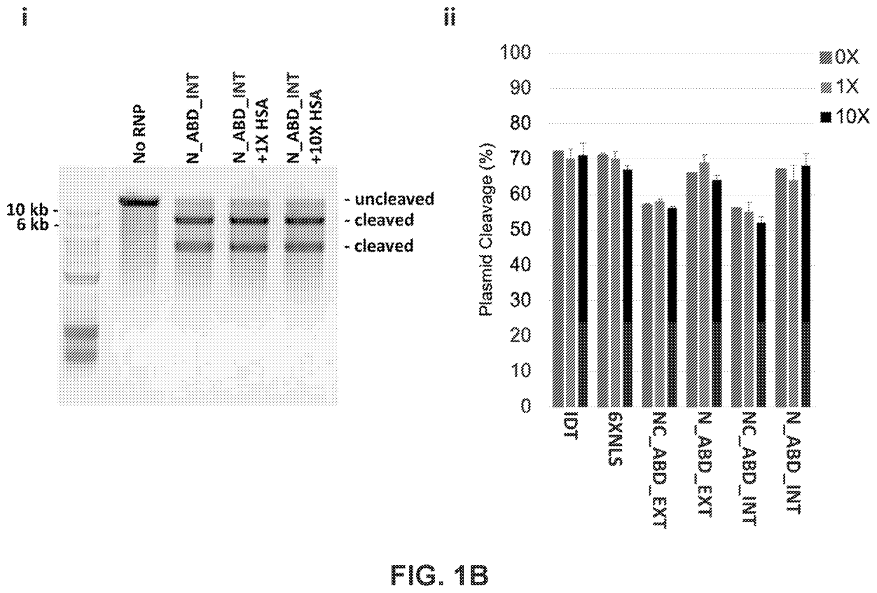

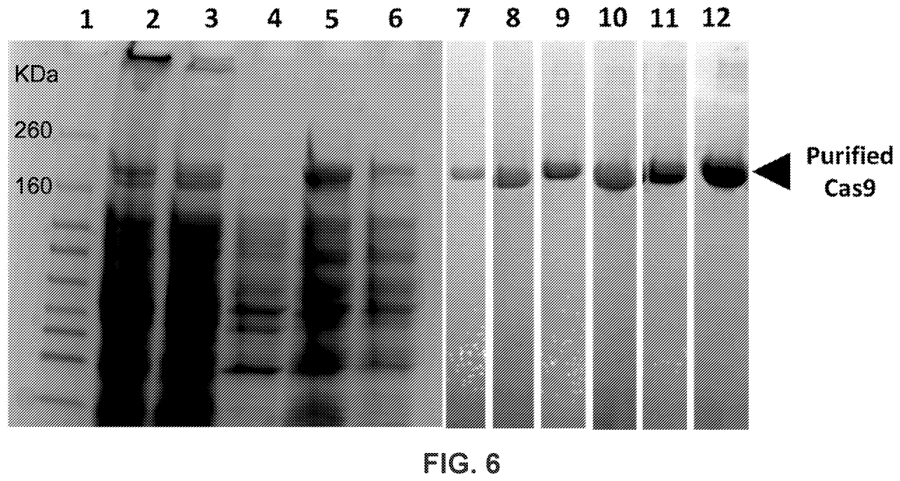

To investigate fusion of an ABD to Cas9, we first produced and purified a library of recombinant Cas9 proteins harboring ABDs at either the N- or both N and C-termini (FIG. 1A). For nuclear entry, three SV40 NLSs in sequence were also added on each side of spCas9. Constructs had the ABDs either internally or externally placed in relation to the NLSs (FIG. 1A). Following purification, we determined protein purity by SDS page showing single bands on elution fraction (FIG. 6). Activity of the different Cas9 variants in making targeted DNA double strand breaks was then tested relative to the parent Cas9 enzyme in vitro using a plasmid cleavage assay (FIG. 1B). In this experiment, RNPs were formed with a gRNA that directs spCas9 to cut a plasmid, resulting in two additional DNA bands on an agarose gel (FIG. 1B(i)). Results show that having one ABD at the N-terminus (either externally or internally in relation to the NLS), exhibits plasmid cleavage activity similar to the parent spCas9 without any ABD (FIG. 1B(ii)). Having an additional ABD at the C-terminus, however, decreases editing efficiency by ~10-15%. Importantly, we have established that the presence of human serum albumin (HSA) does not affect enzymatic activity of spCas9 with ABDs (FIG. 1B (ii). In addition, we determined that adding 6×NLS sequences (3× on each side of spCas9) does not affect activity compared to commercially available enzyme (IDT). Overall, these studies show that we were able to produce recombinant spCas9 proteins harboring ABDs that retain enzymatic activity.

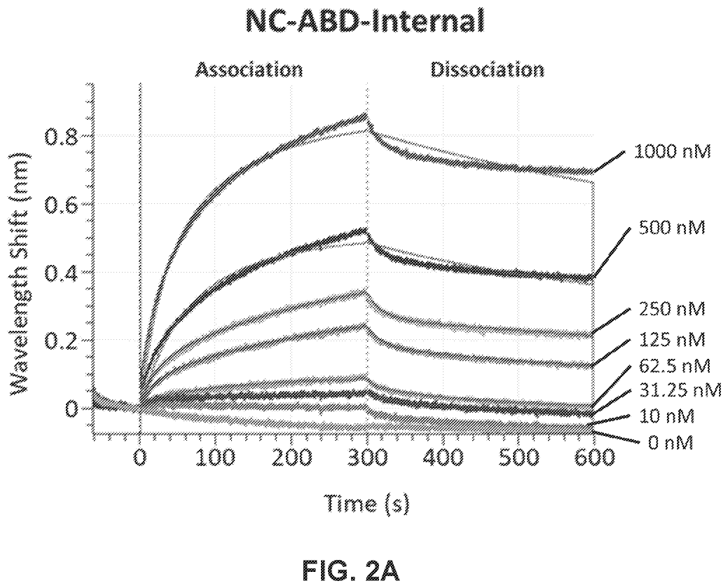

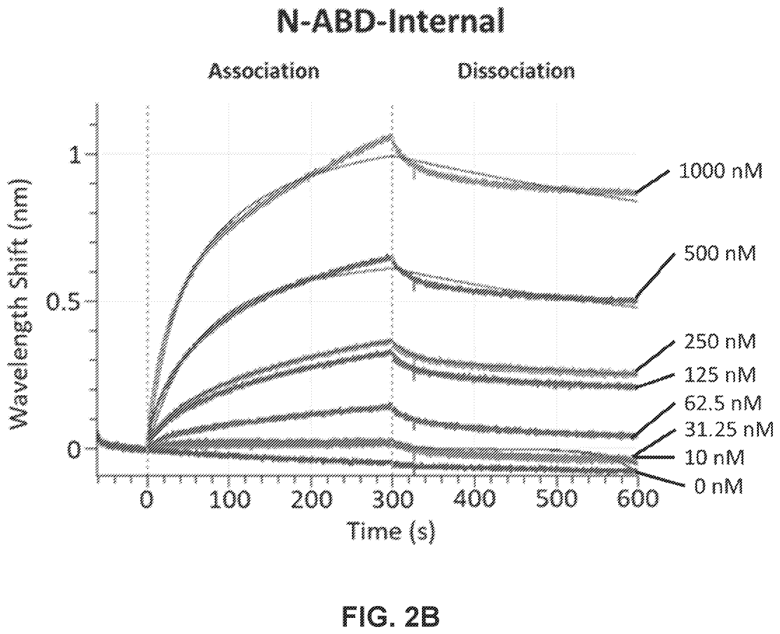

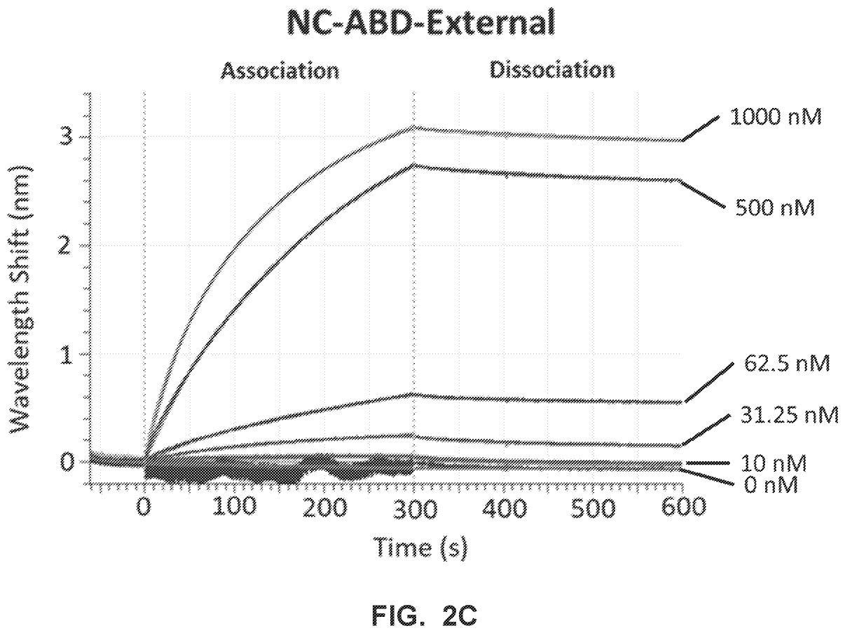

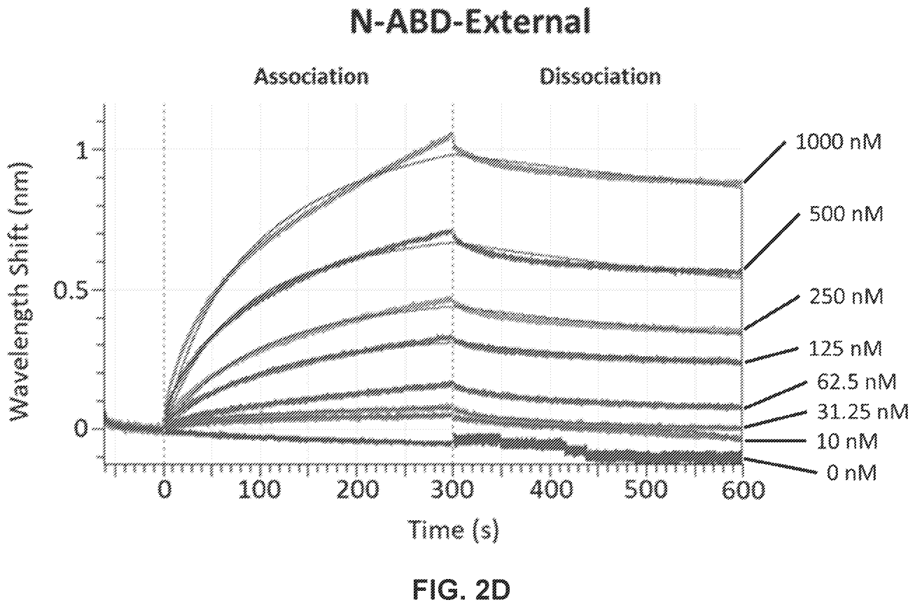

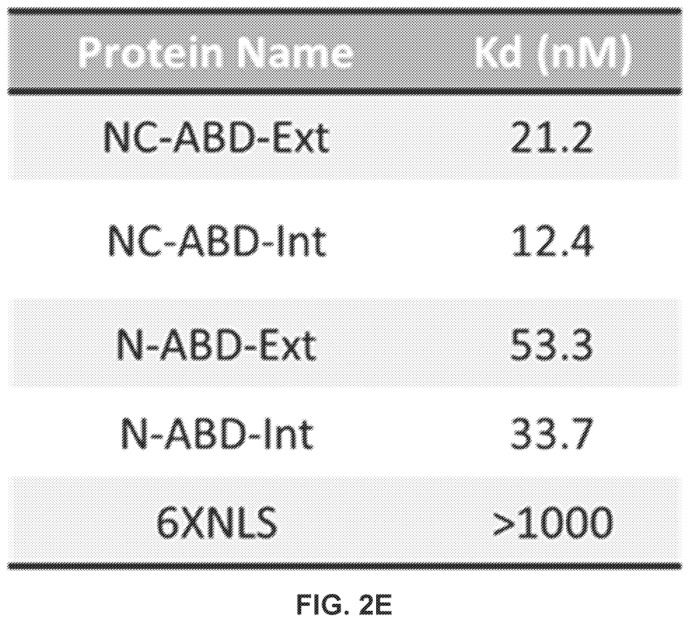

Next, we wanted to determine whether spCas9 fused with ABD(s) binds to albumin. Biolayer interferometry (BLI) was used to measure albumin association and dissociation kinetics of the spCas9 variants (FIG. 2A, FIG. 2B, FIG. 2C, FIG. 2D, and FIG. 2E). spCas9 without ABD did not exhibit albumin binding (K_{D}=>1000 nM). All the spCas9-ABD variants, however, did exhibit albumin binding, although with different affinities. Interestingly, positioning of the ABD in relation to the NLS (externally versus internally), affected albumin binding affinity (N-ABD-Cas9 internal K_{D}=33.7 nM and N-ABD-Cas9 external K_{D}=53.3 nM).

Additionally, when having an ABD at both the N and C-terminus, the ABD internally located seems to have higher albumin binding affinity (NC-ABD-internal K_{D}=12.4 nM versus NC-ABD-external K_{D}=21.2 nM). The BLI measurements confirm that the ABD is functional when fused to a variety of sites on Cas9.

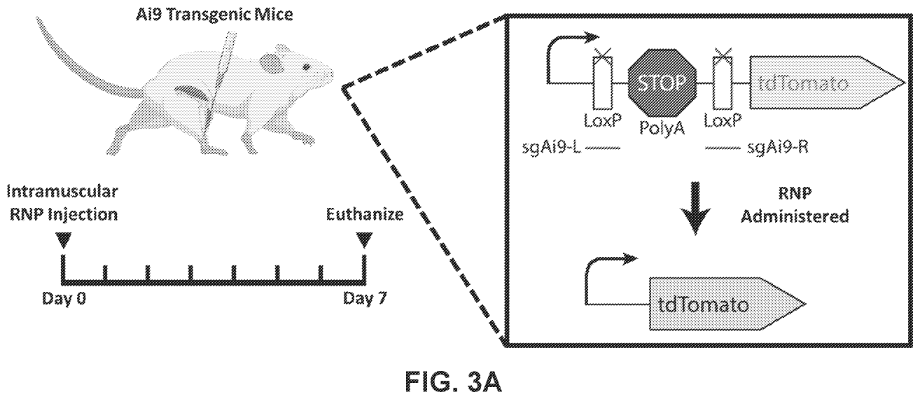

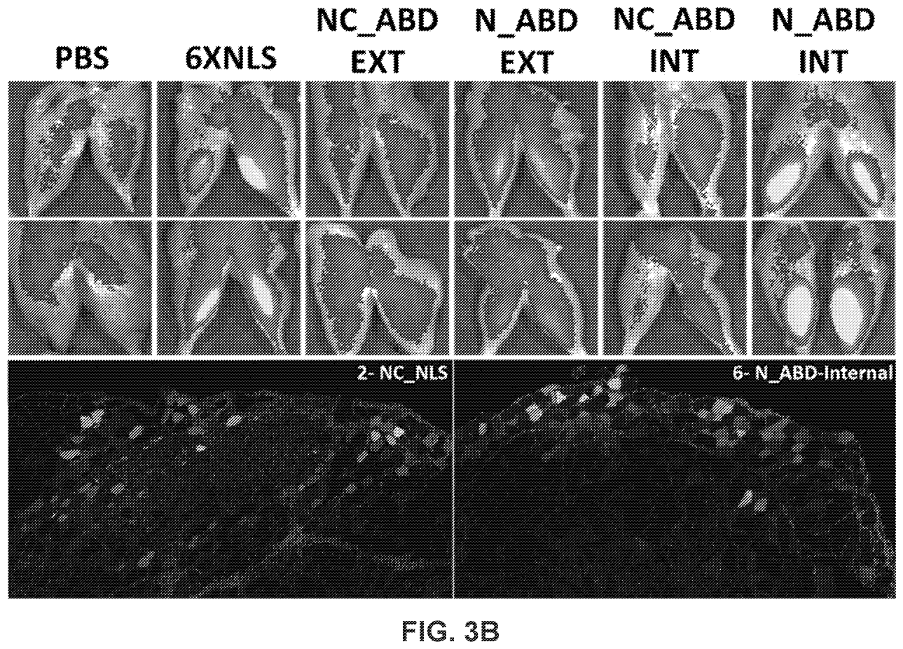

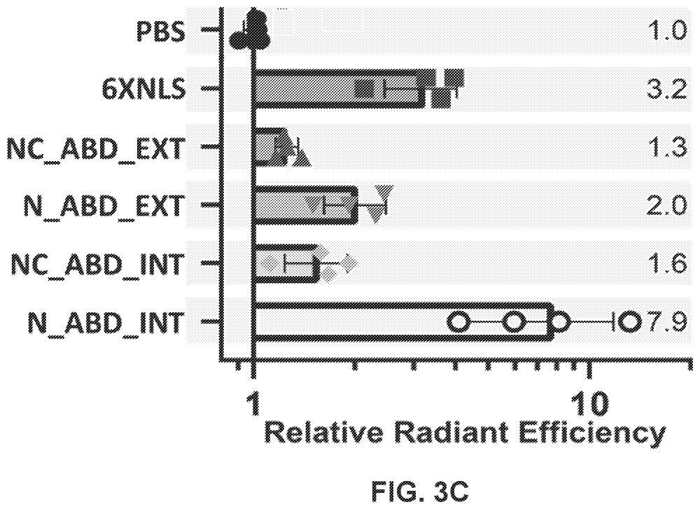

We next screened the gene editing activity of our recombinant Cas9 protein-based RNPs in vivo upon intramuscular injection (IM) into the tibialis anterior (TA) muscle of Ai9 mice. The genomic DNA of Ai9 mice harbors an Ai9 cassette where the DNA encoding a fluorescent tdTomato protein is proceeded by a stop codon flanked by LoxP sites. Delivery of RNPs targeting the LoxP sites creates double strand breaks that cut out the stop codon and activate constitutive expression of tdTomato (FIG. 3A). The N-ABD-internal Cas9 exhibited the highest editing efficiency as measured by IVIS based on tissue tdTomato epifluorescence signal (FIG. 3B and FIG. 3C). Interestingly, the 6×NLS Cas9 without any albumin binding capabilities also exhibited tdTomato turn-on, albeit at a lower efficiency compared to the ABD-Cas9 leading candidate (3.2 versus 7.9 relative fluorescence). We corroborated IVIS quantification with muscle histology, which showed that there is clear tdTomato turn-on in individual muscle fibers.

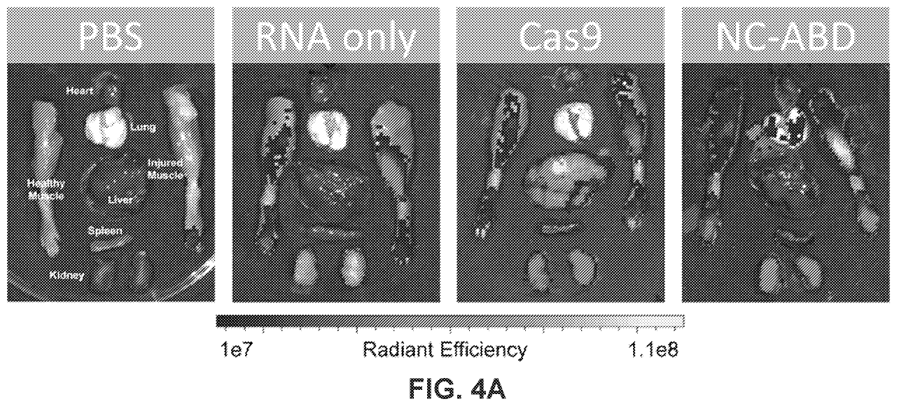

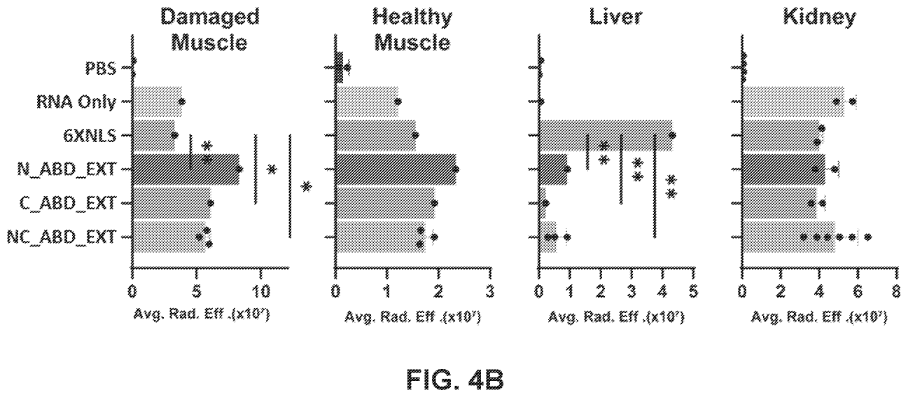

We next tested the ability of albumin binding conjugates to increase delivery and editing activity in inflamed tissue. As a first step, different iterations of RNPs with and without albumin binding capabilities were delivered via intravenous injection following BaCl₂ induced muscle injury to the left TA (FIG. 4A). RNPs tested included Cas9 variants with N-ABD-Ext, C-ABD-Ext, and NC-ABD-Ext. Intravenous administration of saline served as a baseline for tissue autofluorescence, and administration of guide RNA without Cas9 protein acted as a control. The free RNA (“RNA-Only”) accumulated primarily in the kidney and muscle, with minimal liver fluorescence (FIG. 4B). Cas9 without albumin binding capabilities (6×NLS) accumulated primarily in the liver. Notably, the three different ABD-Cas9 proteins (N-ABD-Ext, C-ABD-Ext, and NC-ABD-Ext) were themselves similar, yet differed significantly from no ABD-Cas9 (6×NLS). Specifically, the ABD-Cas9 had greatly reduced liver accumulation (~80% reduction) and significantly enhanced accumulation in the injured muscle (~100% increase). These experiments show that, across the board, the ABD-containing spCas9 RNPs biodistributed more to inflamed muscle and less to the liver compared to the parent spCas9.

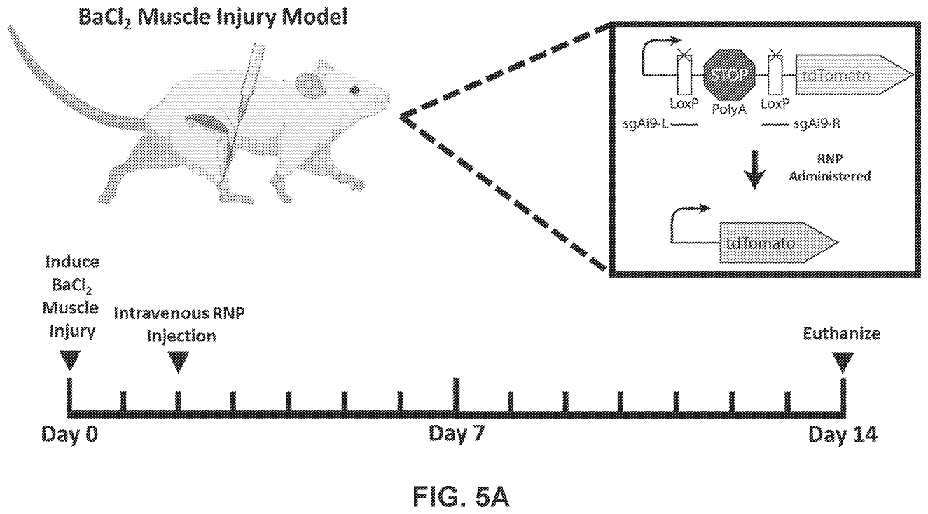

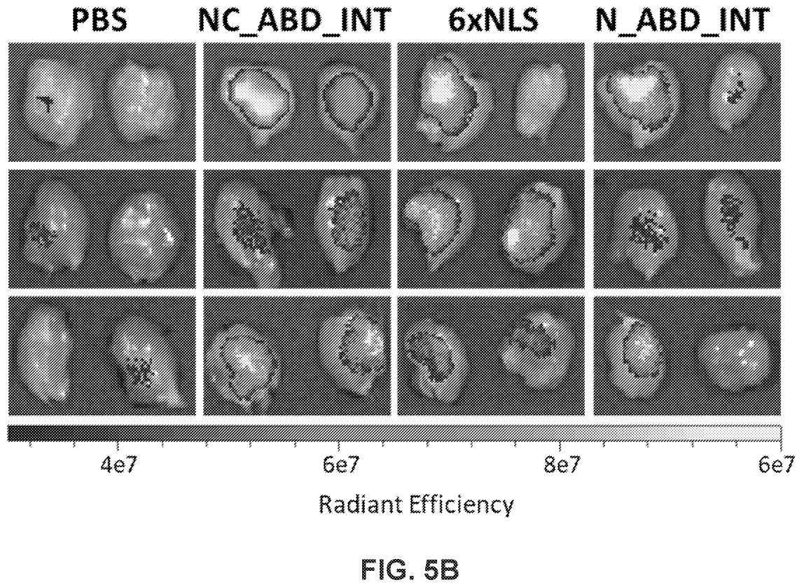

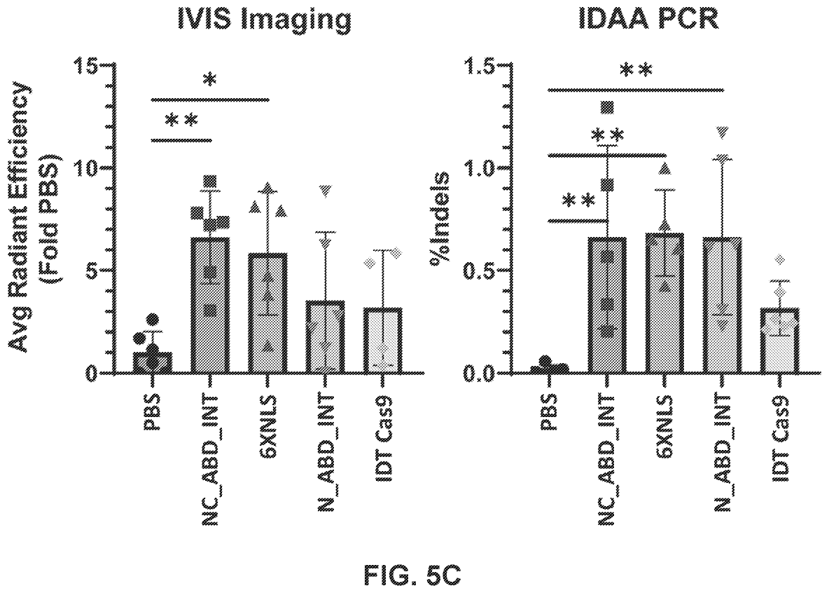

Finally, we assessed in vivo gene editing of the inflamed (BaCl₂-injected) TA muscle of Ai9 mice following intravenous administration of RNPs comprising of Cas9 variants including N-ABD external, N-ABD internal, NC-ABD external, and NC-ABD internal (FIG. 5A). The gene editing activity was again measured based on tdTomato turn-on using IVIS and with a method for indel detection by amplicon analysis (IDAA), a tri-primer amplicon labelling and DNA capillary electrophoresis indel detection. These results show increased gene editing in injured skeletal muscle of the NC-ABD-internal conjugate compared to 6×NLS Cas9 and N-ABD-internal measured by both IVIS and IDAA (FIG. 5B and FIG. 5C).

We have described a technology that leverages albumin binding for establishing a carrier-free gene editing strategy. Albumin-binding can target Cas9 protein to inflamed skeletal muscle. Once albumin concentrates Cas9 in the tissue of interest (inflamed skeletal muscle), nuclear localization signals enable cellular uptake and nuclear delivery for editing. Overall, this disclosure describes a delivery platform for carrier-free CRISPR-Cas9 editing.

TABLE 1
Example Recombinant Cas9 Proteins

| | | CL7 | HRV3C | N- | Linker | N- | Linker | | Linker | C- | Linker | C- | Linker | HIS |
|---|---|---|---|---|---|---|---|---|---|---|---|---|---|---|
| Num | Construct | tag | Site | ABD | 1 | NLS | 2 | Cas9 | 3 | NLS | 4 | ABD | 5 | tag |
| 1 | 6XNLS-Cas9 | X | X | | | X | | X | | X | | | X | X |
| 2 | NC_ABD_Ca | X | X | X | X | X | | X | | X | X | X | X | X |
| | s9_External | | | | | | | | | | | | | |
| 3 | N_ABD_Cas | X | X | X | X | X | | X | | X | | | X | X |
| | 9_External | | | | | | | | | | | | | |
| 4 | NC_ABD_Ca | X | X | X | X | X | X | X | X | X | X | X | X | X |
| | s9_Internal | | | | | | | | | | | | | |
| 5 | N_ABD_Cas | X | X | X | X | X | X | X | | X | | | X | X |
| | 9_Internal | | | | | | | | | | | | | |
| 6 | C_ABD_Cas | X | X | | | X | | X | | X | X | X | X | X |
| | 9_External | | | | | | | | | | | | | |

It is understood that the foregoing detailed description and accompanying examples are merely illustrative and are not to be taken as limitations upon the scope of the disclosure.

Various changes and modifications to the disclosed embodiments will be apparent to those skilled in the art. Such changes and modifications, including without limitation those relating to the structures, substituents, derivatives, intermediates, syntheses, compositions, formulations, or methods of use of the technology, may be made without departing from the spirit and scope thereof.

For reasons of completeness, various aspects of the disclosure are set out in the following numbered clauses:

Clause 1. A recombinant protein comprising: an albumin binding domain; a nuclear localization domain; and a Cas9 domain.

Clause 2. The recombinant protein of clause 1, comprising a linker between the albumin binding domain and the nuclear localization domain.

Clause 3. The recombinant protein of clause 1 or 2, comprising more than one albumin binding domain, more than one nuclear localization domain, or a combination thereof.

Clause 4. The recombinant protein of any one of clauses 1-3, comprising 1 to 4 albumin binding domains.

Clause 5. The recombinant protein of any one of clauses 1-4, wherein the albumin binding domain comprises an amino acid sequence of SEQ ID NO:20.

Clause 6. The recombinant protein of any one of clauses 1-5, comprising 1 to 10 nuclear localization domains.

Clause 7. The recombinant protein of any one of clauses 1-6, wherein the nuclear localization domain comprises an amino acid sequence of SEQ ID NO:21.

Clause 8. The recombinant protein of any one of clauses 1-7, wherein the Cas9 domain comprises an amino acid sequence of SEQ ID NO:29.

Clause 9. The recombinant protein of any one of clauses 2-8, wherein the linker comprises an amino acid sequence of SEQ ID NO:22.

Clause 10. The recombinant protein of any one of clauses 1-9, wherein the albumin binding domain is between the Cas9 domain and the nuclear localization domain.

Clause 11. The recombinant protein of any one of clauses 2-10, comprising, in a N-terminus to a C-terminus direction: the nuclear localization domain; the linker; the albumin binding domain; and the Cas 9 domain.

Clause 12. The recombinant protein of clause 11, comprising an amino acid sequence that is at least 95% identical to any one of SEQ ID NO:26 and SEQ ID NO:27.

Clause 13. The recombinant protein of clause 12, comprising an amino acid sequence that is at least 99% identical to any one of SEQ ID NO:26 and SEQ ID NO:27.

Clause 14. The recombinant protein of any one of clauses 2-11, comprising, in a N-terminus to a C-terminus direction: the nuclear localization domain; the linker; the albumin binding domain; the Cas 9 domain; the albumin binding domain; the linker; and the nuclear localization domain.

Clause 15. The recombinant protein of clause 14, comprising an amino acid sequence that is at least 95% identical to SEQ ID NO:26.

Clause 16. The recombinant protein of clause 15, comprising an amino acid sequence that is at least 99% identical to SEQ ID NO:26.

Clause 17. The recombinant protein of any one of clauses 1-16, having a molecular weight of about 100 kDa to about 300 kDa.

Clause 18. The recombinant protein of any one of clauses 1-17, having a binding affinity (K_{D}) to albumin of less than 50 nM.

Clause 19. The recombinant protein of any one of clauses 1-10, comprising an amino acid sequence that is at least 95% identical to any one of SEQ ID NO:24, SEQ ID NO:25, SEQ ID NO: 26, SEQ ID NO:27, and SEQ ID NO:28.

Clause 20. The recombinant protein of any one of clauses 1-10, comprising an amino acid sequence that is at least 99% identical to any one of SEQ ID NO:24, SEQ ID NO:25, SEQ ID NO: 26, SEQ ID NO:27, and SEQ ID NO:28.

Clause 21. A recombinant protein comprising a multi-domain structure of formula (I)

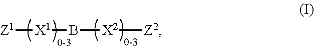

wherein: B is a Cas9 domain; X¹ and X², at each occurrence, are each independently a linker, an albumin binding domain, or a nuclear localization domain; Z¹ and Z² are each independently an albumin binding domain or a nuclear localization domain, and the multi-domain structure of formula (I) has at least one albumin binding domain and at least one nuclear localization domain.

Clause 22. The recombinant protein of clause 21, wherein the albumin binding domain comprises an amino acid sequence of SEQ ID NO:20.

Clause 23. The recombinant protein of clause 21 or 22, wherein the nuclear localization domain comprises an amino acid sequence of SEQ ID NO: 21.

Clause 24. The recombinant protein of any one of clauses 21-23, wherein the Cas9 domain comprises an amino acid sequence of SEQ ID NO:29.

Clause 25. The recombinant protein of any one of clauses 21-24, wherein the linker comprises an amino acid sequence of SEQ ID NO:22.

Clause 26. A composition comprising: the recombinant protein of any one of clauses 1-25; and one or more guide RNAs (gRNA).

Clause 27. The composition of clause 26, wherein the gRNA is a single-guide RNA (sgRNA), the sgRNA comprising a crRNA attached to a tracrRNA.

Clause 28. A method of modulating gene expression in a subject in need thereof, the method comprising administering to the subject a therapeutically effective amount of the composition of clause 26 or 27.

Clause 29. A method of treating a genetic disease in a patient in need thereof, the method comprising administering to the patient a therapeutically effective amount of a composition, the composition comprising: a recombinant protein of any one of clauses 1-25; and one or more gRNAs.

Clause 30. The method of clause 29, wherein the genetic disease is selected from any one of Duchenne muscular dystrophy (DMD), Limb Girdle muscular dystrophy (LGMD), myotonic dystrophy, Charcot-Marie Tooth Syndrome (CMT), Spinal Cerebellar Ataxia, and Huntington's disease.

Clause 31. A composition for use in the treatment of a genetic disease, the composition comprising: a recombinant protein of any one of clauses 1-25; and one or more gRNAs.

Clause 32. The composition for use of clause 31, wherein the genetic disease is selected from any one of Duchenne muscular dystrophy (DMD), Limb Girdle muscular dystrophy (LGMD), myotonic dystrophy, Charcot-Marie Tooth Syndrome (CMT), Spinal Cerebellar Ataxia, and Huntington's disease.

Clause 33. A method of modifying a targeted gene associated with disease in a patient, the method comprising administering to the patient a therapeutically effective amount of a composition, the composition comprising: the recombinant protein of any one of clauses 1-25; and one or more gRNAs.

Clause 34. The method of clause 33, wherein the targeted gene is selected from any one of a dystrophin (DMD) gene, a calpain 3 (CAPN3) gene, a dysferlin (DYSF) gene, an alpha-sarcoglycan (SGCA) gene, a beta-sarcoglycan (SGCB) gene, a gamma-sarcoglycan (SGCG) gene, a delta-sarcoglycan gene (SGCD) gene, a telethonin (TCAP) gene, a fukutin-related protein (FKRP) gene, an anoctamin 5 (ANO5) gene, a DM1 gene, a DM2 gene, a neurotrophin 3 (NT3) gene, and a huntingtin (HTT) gene.

Clause 35. The method of clause 33 or 34, wherein the targeted gene is a human gene.

Clause 36. The recombinant protein of any one of clauses 1-4, 6-21, and 23-25, wherein the albumin binding domain comprises an amino acid sequence selected from the group consisting of SEQ ID NO:20, SEQ ID NO:30, SEQ ID NO:31, SEQ ID NO:32, SEQ ID NO: 33, SEQ ID NO:34, SEQ ID NO:35, and combination thereof.

Clause 37. The recombinant protein of any one of clauses 1-6, 8-22, and 24-25, wherein the nuclear localization domain comprises an amino acid sequence selected from the group consisting of SEQ ID NO:21, SEQ ID NO:36-SEQ ID NO:46, and a combination thereof.

Clause 38. The recombinant protein of any one of clauses 1-8 and 10-24, wherein the linker comprises an amino acid sequence selected from the group consisting of SEQ ID NO:22, SEQ ID NO:47-SEQ ID NO:57, and a combination thereof.

### Sequences

Nucleotide Sequences:

CL7 tag:
atgagcaaaagcaatgaaccgggtaaagcaaccggtgaaggtaaaccggttaataacaaatggctgaacaatgccggtaaagat
ctgggtagtccggttccggatcgtattgcaaataaactgcgtgataaagaattcgagagcttcgatgattttcgtgaaaccttttgggaag
aagttagcaaagatcctgaactgagcaaacagtttagccgcaataacaatgatcgtatgaaagttggtaaagcaccgaaaacacgt
acccaggatgttagcggtaaacgtacctcatttgaactgaatcatcagaaaccgattgaacagaatggtggcgtttatgatatggataa
cattagcgttgttaccccgaaacgcaacattgatattgaaggggtggtggtggttct (SEQ ID NO: 1)
HRV 3C Site:
ttggaggttttgttccagggtcca (SEQ ID NO: 2)
N-ABD:
cttaaagaagctaaagagaaagcgatcgaagaattgaaaaaggctggcattacgtccgattattattttgatttgattaacaaggcaaa
gaccgtcgagggagtcaatgcccttaaagatgaaatcttaaaagct (SEQ ID NO: 3)
Linker 1;
gatcaggtggaggaggttcgggcggtggtgggagcgggggtggcggttct (SEQ ID NO: 4)
N-NLS:
ccgaaaaagaaacggaaagttgatcctaaaaagaagcgtaaggtagatccaaagaaaaaacgcaaagtt (SEQ ID NO: 5)
Linker 2:
ggctcgggtggaggcggttcgggcgggggtggctctgggggtggcggatct (SEQ ID NO: 6)
Cas 9:
atggataagaaatactcaataggcttagatatcggcacaaatagcgtcggatgggcggtgatcactgatgaatataaggttccgtcta
aaaagttcaaggttctgggaaatacagaccgccacagtatcaaaaaaaatcttataggggctcttttatttgacagtggagagacagc
ggaagcgactcgtctcaaacggacagctcgtagaaggtatacacgtcggaagaatcgtatttgttatctacaggagattttttcaaatga
gatggcgaaagtagatgatagtttctttcatcgacttgaagagtcttttttggtggaagaagacaagaagcatgaacgtcatcctatttttg
gaaatatagtagatgaagttgcttatcatgagaaatatccaactatctatcatctgcgaaaaaaattggtagattctactgataaagcgg
atttgcgcttaatctatttggccttagcgcatatgattaagtttcgtggtcattttttgattgagggagatttaaatcctgataatagtgatgt
ggacaaactatttatccagttggtacaaacctacaatcaattatttgaagaaaaccctattaacgcaagtggagtagatgctaaagcgattctt
tctgcacgattgagtaaatcaagacgattagaaaatctcattgctcagctccccggtgagaagaaaaatggcttatttgggaatctcatt
gctttgtcattgggtttgacccctaattttaaatcaaattttgatttggcagaagatgctaaattacagctttcaaaagatacttacgatgatg
atttagataatttattggcgcaaattggagatcaatatgctgatttgtttttggcagctaagaatttatcagatgctattttactttcagatat
cctaagagtaaatactgaaataactaaggctcccctatcagcttcaatgattaaacgctacgatgaacatcatcaagacttgactcttttaaa
agctttagttcgacaacaacttccagaaaagtataaagaaatcttttttgatcaatcaaaaaacggatatgcaggttatattgatggggg
agctagccaagaagaattttataaatttatcaaaccaattttagaaaaaatggatggtactgaggaattattggtgaaactaaatcgtga
agatttgctgcgcaagcaacggacctttgacaacggctctattccccatcaaattcacttgggtgagctgcatgctattttgagaagaca
agaagacttttatccatttttaaaagacaatcgtgagaagattgaaaaaatcttgacttttcgcattccttattatgttggtccattggcgcgt
ggcaatagtcgttttgcatggatgactcggaagtctgaagaaacaattaccccatggaattttgaagaagttgtcgataaaggtgcttcag
ctcaatcatttattgaacgcatgacaaactttgataaaaatcttccaaatgaaaaagtactaccaaaacatagtttgctttatgagtatttta
cggtttataacgaattgacaaaggtcaaatatgttactgaaggaatgcgaaaaccagcatttctttcaggtgaacagaagaaagccat
tgttgatttactcttcaaaacaaatcgaaaagtaaccgttaagcaattaaaagaagattatttcaaaaaaatagaatgttttgatagtgttg
aaatttcaggagttgaagatagatttaatgcttcattaggtacctaccatgatttgctaaaaattattaaagataaagattttttggataatga
agaaaatgaagatatcttagaggatattgttttaacattgaccttatttgaagatagggagatgattgaggaaagacttaaaacatatgct
cacctctttgatgataaggtgatgaaacagcttaaacgtcgccgttatactggttggggacgtttgtctcgaaaattgattaatggtattag
ggataagcaatctggcaaaacaatattagattttttgaaatcagatggttttgccaatcgcaattttatgcagctgatccatgatgatagttt
gacatttaaagaagacattcaaaaagcacaagtgtctggacaaggcgatagtttacatgaacatattgcaaatttagctggtagccctg
ctattaaaaaaggtattttacagactgtaaaagttgttgatgaattggtcaaagtaatggggcggcataagccagaaaatatcgttattg
aaatggcacgtgaaaatcagacaactcaaaagggccagaaaaattcgcgagagcgtatgaaacgaatcgaagaaggtatcaaa
gaattaggaagtcagattcttaaagagcatcctgttgaaaatactcaattgcaaaatgaaaagctctatctctattatctccaaaatggaa
gagacatgtatgtggaccaagaattagatattaatcgtttaagtgattatgatgtcgatcacattgttccacaaagtttccttaaagacgatt
caatagacaataaggtcttaacgcgttctgataaaaatcgtggtaaatcggataacgttccaagtgaagaagtagtcaaaaagatga
aaaactattggagacaacttctaaacgccaagttaatcactcaacgtaagtttgataatttaacgaaagctgaacgtggaggtttgagt
gaacttgataaagctggttttatcaaacgccaattggttgaaactcgccaaatcactaagcatgtggcacaaattttggatagtcgcatg
aatactaaatacgatgaaaatgataaacttattcgagaggttaaagtgattaccttaaaatctaaattagtttctgacttccgaaaagattt
ccaattctataaagtacgtgagattaacaattaccatcatgcccatgatgcgtatctaaatgccgtcgttggaactgctttgattaagaaat
atccaaaacttgaatcggagtttgtctatggtgattataaagtttatgatgttcgtaaaatgattgctaagtctgagcaagaaataggcaaa
gcaaccgcaaaatatttttttactctaatatcatgaacttcttcaaaacagaaattacacttgcaaatggagagattcgcaaacgccctc
taatcgaaactaatggggaaactggagaaattgtctgggataaagggcgagattttgccacagtgcgcaaagtattgtccatgcccca
agtcaatattgtcaagaaaacagaagtacagacaggcggattctccaaggagtcaattttaccaaaaagaaattcggacaagcttatt
gctcgtaaaaaagactgggatccaaaaaaatatggtggttttgatagtccaacggtagcttattcagtcctagtggttgctaaggtggaa
aaagggaaatcgaagaagttaaaatccgttaaagagttactagggatcacaattatggaaagaagttcctttgaaaaaaatccgattg
actttttagaagctaaaggatataaggaagttaaaaaagacttaatcattaaactacctaaatatagtctttttgagttagaaaacggtcgt
aaacggatgctggctagtgccggagaattacaaaaaggaaatgagctggctctgccaagcaaatatgtgaattttttatatttagctagt
cattatgaaaagttgaagggtagtccagaagataacgaacaaaaacaattgtttgtggagcagcataagcattatttagatgagattat
tgagcaaatcagtgaattttctaagcgtgttattttagcagatgccaatttagataaagttcttagtgcatataacaaacatagagacaaa
ccaatacgtgaacaagcagaaaatattattcatttatttacgttgacgaatcttggagctcccgctgcttttaaatattttgatacaacaattg
atcgtaaacgatatacgtctacaaaagaagttttagatgccactcttatccatcaatccatcactggtctttatgaaacacgcattgatttga
gtcagctaggaggtgac (SEQ ID NO: 7)
Linker 5:
ggaagcggcggagggggcagtggcgggggtggaagtggtggaggtggcagc (SEQ ID NO: 8)
C-NLS:
ccgaagaaaaaacgtaaagtagaccctaagaaaaaacgcaaggtagaccctaagaaaaagcggaaagtc (SEQ
ID NO: 9)
Linker 4:
ggatcaggtggaggaggttcgggcggtggtgggagcgggggtggcggttct (SEQ ID NO: 10)
C-ABD:
ttaaagaagctaaagagaaagcgatcgaagaattgaaaaaggctggcattacgtccgattattattttgatttgattaacaaggcaaag
accgtcgagggagtcaatgcccttaaagatgaaatcttaaaagct (SEQ ID NO: 11)
Linker 5:
ggtggaggaggttcgggcggtggtgggagcgggggtggcggttct (SEQ ID NO: 12)
6X HIS:
catcaccaccaccatcac (SEQ ID NO: 13)

TABLE 2
PCR primers used in IDAA

| | FWD | REV |
|---|---|---|
| Set 1 | IDAA1.F: ttcggcttctggcgtgtg | IDAA2.R: |
| | (SEQ ID NO: 14) | AGCTGACCGGCAGCAAAATTGctttgatg |
| | | acctcctcgcccttg (SEQ ID NO: 15) |
| Set 2 | IDAA2.F: | IDAA2.R: cctcctcgcccttgctc |
| | AGCTGACCGGCAGCAAAATTGcctctgct | (SEQ ID NO: 17) |
| | aaccatgttcatgcc (SEQ ID NO: 16) | |
| Set 3 | IDAA3.F: | IDAA2.R: cctcctcgcccttgctc |
| | AGCTGACCGGCAGCAAAATTGctgggc | (SEQ ID NO: 19) |
| | aacgtgctggttattg (SEQ ID NO: 18) | |

### Amino Acid Sequences:

ABD:
(SEQ ID NO: 20)
LKEAKEKAIEELKKAGITSDYYFDLINKAKTVEGVNALKDEILKA
SV40 NLS;
(SEQ ID NO: 21)
PKKKRKV
Linker:
(SEQ ID NO: 22)
(GGGGS)₃

| | Construct | Sequence |
|---|---|---|
| 1 | 6xNLS-Cas9 | GPPKKKRKVDPKKKRKVDPKKKRKVMDKKYSIGLDIGTNSVGWAVITDEYKVPSKKFKVLGN |
| | (SEQ ID | TDRHSIKKNLIGALLFDSGETAEATRLKRTARRRYTRRKNRICYLQEIFSNEMAKVDDSFFHRL |
| | NO: 23) | EESFLVEEDKKHERHPIFGNIVDEVAYHEKYPTIYHLRKKLVDSTDKADLRLIYLALAHMIKFRG |
| | | HFLIEGDLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQYADLFLAA |
| | | KNLSDAILLSDILRVNTEITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTFDNGSIPHQIHLGELHAIL |
| | | RRQEDFYPFLKDNREKIEKILTFRIPYYVGPLARGNSRFAWMTRKSEETITPWNFEEVVDKGA |
| | | SAQSFIERMTNFDKNLPNEKVLPKHSLLYEYFTVYNELTKVKYVTEGMRKPAFLSGEQKKAIV |
| | | DLLFKTNRKVTVKQLKEDYFKKIECFDSVEISGVEDRFNASLGTYHDLLKIIKDKDFLDNEENED |
| | | ILEDIVLTLTLFEDREMIEERLKTYAHLFDDKVMKQLKRRRYTGWGRLSRKLINGIRDKQSGKT |
| | | ILDFLKSDGFANRNFMQLIHDDSLTFKEDIQKAQVSGQGDSLHEHIANLAGSPAIKKGILQTV |
| | | KVVDELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLTRSDKNR |
| | | GKSDNVPSEEVVKKMKNYWRQLLNAKLITQRKFDNLTKAERGGLSELDKAGFIKRQLVETR |
| | | QITKHVAQILDSRMNTKYDENDKLIREVKVITLKSKLVSDFRKDFQFYKVREINNYHHAHDAY |
| | | LNAVVGTALIKKYPKLESEFVYGDYKVYDVRKMIAKSEQEIGKATAKYFFYSNIMNFFKTEITL |
| | | ANGEIRKRPLIETNGETGEIVWDKGRDFATVRKVLSMPQVNIVKKTEVQTGGFSKESILPKRN |
| | | SDKLIARKKDWDPKKYGGFDSPTVAYSVLVVAKVEKGKSKKLKSVKELLGITIMERSSFEKNPI |
| | | DFLEAKGYKEVKKDLIIKLPKYSLFELENGRKRMLASAGELQKGNELALPSKYVNFLYLASHYE |
| | | KLKGSPEDNEQKQLFVEQHKHYLDEIIEQISEFSKRVILADANLDKVLSAYNKHRDKPIREQAE |
| | | NIIHLFTLTNLGAPAAFKYFDTTIDRKRYTSTKEVLDATLIHQSITGLYETRIDLSQLGGDPKKKR |
| | | KVDPKKKRKVDPKKKRKVGSGGGGSGGGGSGGGGSHHHHHH |
| 2 | NC_ABD_Cas9_ | GPLKEAKEKAIEELKKAGITSDYYFDLINKAKTVEGVNALKDEILKAGSGGGGSGGGGSGGGG |
| | External | SPKKKRKVDPKKKRKVDPKKKRKVMDKKYSIGLDIGTNSVGWAVITDEYKVPSKKFKVLGNT |
| | (SEQ ID | DRHSIKKNLIGALLFDSGETAEATRLKRTARRRYTRRKNRICYLQEIFSNEMAKVDDSFFHRLE |
| | NO: 24) | ESFLVEEDKKHERHPIFGNIVDEVAYHEKYPTIYHLRKKLVDSTDKADLRLIYLALAHMIKFRGH |
| | | FLIEGDLNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYADLFLAAK |
| | | NLSDAILLSDILRVNTEITKAPLSASMIKRYDEHHQDLTLLKALVRQQLPEKYKEIFFDQSKNGY |
| | | AGYIDGGASQEEFYKFIKPILEKMDGTEELLVKLNREDLLRKQRTFDNGSIPHQIHLGELHAILR |
| | | RQEDFYPFLKDNREKIEKILTFRIPYYVGPLARGNSRFAWMTRKSEETITPWNFEEVVDKGAS |
| | | AQSFIERMTNFDKNLPNEKVLPKHSLLYEYFTVYNELTKVKYVTEGMRKPAFLSGEQKKAIVD |
| | | LLFKTNRKVTVKQLKEDYFKKIECFDSVEISGVEDRFNASLGTYHDLLKIIKDKDFLDNEENEDIL |
| | | EDIVLTLTLFEDREMIEERLKTYAHLFDDKVMKQLKRRRYTGWGRLSRKLINGIRDKQSGKTIL |
| | | DFLKSDGFANRNFMQLIHDDSLTFKEDIQKAQVSGQGDSLHEHIANLAGSPAIKKGILQTVK |
| | | VVDELVKVMGRHKPENIVIEMARENQTTQKGQKNSRERMKRIEEGIKELGSQILKEHPVENT |
| | | QLQNEKLYLYYLQNGRDMYVDQELDINRLSDYDVDHIVPQSFLKDDSIDNKVLTRSDKNRGK |
| | | SDNVPSEEVVKKMKNYWRQLLNAKLITQRKFDNLTKAERGGLSELDKAGFIKRQLVETRQIT |
| | | KHVAQILDSRMNTKYDENDKLIREVKVITLKSKLVSDFRKDFQFYKVREINNYHHAHDAYLNA |
| | | VVGTALIKKYPKLESEFVYGDYKVYDVRKMIAKSEQEIGKATAKYFFYSNIMNFFKTEITLANG |
| | | EIRKRPLIETNGETGEIVWDKGRDFATVRKVLSMPQVNIVKKTEVQTGGFSKESILPKRNSDK |
| | | LIARKKDWDPKKYGGFDSPTVAYSVLVVAKVEKGKSKKLKSVKELLGITIMERSSFEKNPIDFL |
| | | EAKGYKEVKKDLIIKLPKYSLFELENGRKRMLASAGELQKGNELALPSKYVNFLYLASHYEKLK |
| | | GSPEDNEQKQLFVEQHKHYLDEIIEQISEFSKRVILADANLDKVLSAYNKHRDKPIREQAENII |
| | | HLFTLTNLGAPAAFKYFDTTIDRKRYTSTKEVLDATLIHQSITGLYETRIDLSQLGGDPKKKRKV |
| | | DPKKKRKVDPKKKRKVGSGGGGSGGGGSGGGGSLKEAKEKAIEELKKAGITSDYYFDLINKA |
| | | KTVEGVNALKDEILKAGGGGSGGGGSGGGGSHHHHHH |
| 3 | NC_ABD_Cas9_ | GPLKEAKEKAIEELKKAGITSDYYFDLINKAKTVEGVNALKDEILKAGSGGGGSGGGGSGGGG |
| | External | SPKKKRKVDPKKKRKVDPKKKRKVMDKKYSIGLDIGTNSVGWAVITDEYKVPSKKFKVLGNT |
| | (SEQ ID | DRHSIKKNLIGALLFDSGETAEATRLKRTARRRYTRRKNRICYLQEIFSNEMAKVDDSFFHRLE |
| | NO: 25) | ESFLVEEDKKHERHPIFGNIVDEVAYHEKYPTIYHLRKKLVDSTDKADLRLIYLALAHMIKFRGH |
| | | FLIEGDLNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYADLFLAAK |
| | | NLSDAILLSDILRVNTEITKAPLSASMIKRYDEHHQDLTLLKALVRQQLPEKYKEIFFDQSKNGY |
| | | AGYIDGGASQEEFYKFIKPILEKMDGTEELLVKLNREDLLRKQRTFDNGSIPHQIHLGELHAILR |
| | | RQEDFYPFLKDNREKIEKILTFRIPYYVGPLARGNSRFAWMTRKSEETITPWNFEEVVDKGAS |
| | | AQSFIERMTNFDKNLPNEKVLPKHSLLYEYFTVYNELTKVKYVTEGMRKPAFLSGEQKKAIVD |
| | | LLFKTNRKVTVKQLKEDYFKKIECFDSVEISGVEDRFNASLGTYHDLLKIIKDKDFLDNEENEDIL |
| | | EDIVLTLTLFEDREMIEERLKTYAHLFDDKVMKQLKRRRYTGWGRLSRKLINGIRDKQSGKTIL |
| | | DFLKSDGFANRNFMQLIHDDSLTFKEDIQKAQVSGQGDSLHEHIANLAGSPAIKKGILQTVK |
| | | VVDELVKVMGRHKPENIVIEMARENQTTQKGQKNSRERMKRIEEGIKELGSQILKEHPVENT |
| | | QLQNEKLYLYYLQNGRDMYVDQELDINRLSDYDVDHIVPQSFLKDDSIDNKVLTRSDKNRGK |
| | | SDNVPSEEVVKKMKNYWRQLLNAKLITQRKFDNLTKAERGGLSELDKAGFIKRQLVETRQIT |
| | | KHVAQILDSRMNTKYDENDKLIREVKVITLKSKLVSDFRKDFQFYKVREINNYHHAHDAYLNA |
| | | VVGTALIKKYPKLESEFVYGDYKVYDVRKMIAKSEQEIGKATAKYFFYSNIMNFFKTEITLANG |
| | | EIRKRPLIETNGETGEIVWDKGRDFATVRKVLSMPQVNIVKKTEVQTGGFSKESILPKRNSDK |
| | | LIARKKDWDPKKYGGFDSPTVAYSVLVVAKVEKGKSKKLKSVKELLGITIMERSSFEKNPIDFL |
| | | EAKGYKEVKKDLIIKLPKYSLFELENGRKRMLASAGELQKGNELALPSKYVNFLYLASHYEKLK |
| | | GSPEDNEQKQLFVEQHKHYLDEIIEQISEFSKRVILADANLDKVLSAYNKHRDKPIREQAENII |
| | | HLFTLTNLGAPAAFKYFDTTIDRKRYTSTKEVLDATLIHQSITGLYETRIDLSQLGGDPKKKRKV |
| | | DPKKKRKVDPKKKRKVGSGGGGSGGGGSGGGGSHHHHHH |
| 4 | NC_ABD_Cas9_ | GPPKKKRKVDPKKKRKVDPKKKRKVGGGGSGGGGSGGGGSLKEAKEKAIEELKKAGITSDYY |
| | Internal | FDLINKAKTVEGVNALKDEILKAGSGGGGSGGGGSGGGGSMDKKYSIGLDIGTNSVGWAVI |
| | (SEQ ID | TDEYKVPSKKFKVLGNTDRHSIKKNLIGALLFDSGETAEATRLKRTARRRYTRRKNRICYLQEIF |
| | NO: 26) | SNEMAKVDDSFFHRLEESFLVEEDKKHERHPIFGNIVDEVAYHEKYPTIYHLRKKLVDSTDKA |
| | | DLRLIYLALAHMIKFRGHFLIEGDLNPDNSDVDKLFIQLVQTYNQLFEENPINASGVDAKAILS |
| | | ARLSKSRRLENLIAQLPGEKKNGLFGNLIALSLGLTPNFKSNFDLAEDAKLQLSKDTYDDDLDN |
| | | LLAQIGDQYADLFLAAKNLSDAILLSDILRVNTEITKAPLSASMIKRYDEHHQDLTLLKALVRQ |
| | | QLPEKYKEIFFDQSKNGYAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGPLARGNSRFAWMTRKS |
| | | EETITPWNFEEVVDKGASAQSFIERMTNFDKNLPNEKVLPKHSLLYEYFTVYNELTKVKYVTE |
| | | GMRKPAFLSGEQKKAIVDLLFKTNRKVTVKQLKEDYFKKIECFDSVEISGVEDRFNASLGTYH |
| | | DLLKIIKDKDFLDNEENEDILEDIVLTLTLFEDREMIEERLKTYAHLFDDKVMKQLKRRRYTGW |
| | | GRLSRKLINGIRDKQSGKTILDFLKSDGFANRNFMQLIHDDSLTFKEDIQKAQVSGQGDSLHE |
| | | HIANLAGSPAIKKGILQTVKVVDELVKVMGRHKPENIVIEMARENQTTQKGQKNSRERMKR |
| | | IEEGIKELGSQILKEHPVENTQLQNEKLYLYYLQNGRDMYVDQELDINRLSDYDVDHIVPQSF |
| | | LKDDSIDNKVLTRSDKNRGKSDNVPSEEVVKKMKNYWRQLLNAKLITQRKFDNLTKAERGG |
| | | LSELDKAGFIKRQLVETRQITKHVAQILDSRMNTKYDENDKLIREVKVITLKSKLVSDFRKDFQ |
| | | FYKVREINNYHHAHDAYLNAVVGTALIKKYPKLESEFVYGDYKVYDVRKMIAKSEQEIGKATA |
| | | KYFFYSNIMNFFKTEITLANGEIRKRPLIETNGETGEIVWDKGRDFATVRKVLSMPQVNIVKK |
| | | TEVQTGGFSKESILPKRNSDKLIARKKDWDPKKYGGFDSPTVAYSVLVVAKVEKGKSKKLKSV |
| | | KELLGITIMERSSFEKNPIDFLEAKGYKEVKKDLIIKLPKYSLFELENGRKRMLASAGELQKGNE |
| | | LALPSKYVNFLYLASHYEKLKGSPEDNEQKQLFVEQHKHYLDEIIEQISEFSKRVILADANLDKV |
| | | LSAYNKHRDKPIREQAENIIHLFTLTNLGAPAAFKYFDTTIDRKRYTSTKEVLDATLIHQSITGLY |
| | | ETRIDLSQLGGDGSGGGGSGGGGSGGGGSLKEAKEKAIEELKKAGITSDYYFDLINKAKTVEG |
| | | VNALKDEILKAGGGGSGGGGSGGGGSPKKKRKVDPKKKRKVDPKKKRKVGGGGSGGGGS |
| | | GGGGSHHHHHH |
| 5 | N_ABD_Cas9_ | GPPKKKRKVDPKKKRKVDPKKKRKVGGGGSGGGGSGGGGSLKEAKEKAIEELKKAGITSDYY |
| | Internal | FDLINKAKTVEGVNALKDEILKAMDKKYSIGLDIGTNSVGWAVITDEYKVPSKKFKVLGNTDR |
| | (SEQ ID | HSIKKNLIGALLFDSGETAEATRLKRTARRRYTRRKNRICYLQEIFSNEMAKVDDSFFHRLEESF |
| | NO: 27) | LVEEDKKHERHPIFGNIVDEVAYHEKYPTIYHLRKKLVDSTDKADLRLIYLALAHMIKFRGHFLI |
| | | EGDLNPDNSDVDKLFIQLVQTYNQLFEENPINASGVDAKAILSARLSKSRRLENLIAQLPGEKK |
| | | NGLFGNLIALSLGLTPNFKSNFDLAEDAKLQLSKDTYDDDLDNLLAQIGDQYADLFLAAKNLS |
| | | DAILLSDILRVNTEITKAPLSASMIKRYDEHHQDLTLLKALVRQQLPEKYKEIFFDQSKNGYAG |
| | | YIDGGASQEEFYKFIKPILEKMDGTEELLVKLNREDLLRKQRTFDNGSIPHQIHLGELHAILRRQ |
| | | EDFYPFLKDNREKIEKILTFRIPYYVGPLARGNSRFAWMTRKSEETITPWNFEEVVDKGASAQ |
| | | SFIERMTNFDKNLPNEKVLPKHSLLYEYFTVYNELTKVKYVTEGMRKPAFLSGEQKKAIVDLLF |
| | | KTNRKVTVKQLKEDYFKKIECFDSVEISGVEDRFNASLGTYHDLLKIIKDKDFLDNEENEDILED |
| | | IVLTLTLFEDREMIEERLKTYAHLFDDKVMKQLKRRRYTGWGRLSRKLINGIRDKQSGKTILDF |
| | | LKSDGFANRNFMQLIHDDSLTFKEDIQKAQVSGQGDSLHEHIANLAGSPAIKKGILQTVKVV |
| | | DELVKVMGRHKPENIVIEMARENQTTQKGQKNSRERMKRIEEGIKELGSQILKEHPVENTQL |
| | | QNEKLYLYYLQNGRDMYVDQELDINRLSDYDVDHIVPQSFLKDDSIDNKVLTRSDKNRGKSD |
| | | NVPSEEVVKKMKNYWRQLLNAKLITQRKFDNLTKAERGGLSELDKAGFIKRQLVETRQITKH |
| | | VAQILDSRMNTKYDENDKLIREVKVITLKSKLVSDFRKDFQFYKVREINNYHHAHDAYLNAVV |
| | | GTALIKKYPKLESEFVYGDYKVYDVRKMIAKSEQEIGKATAKYFFYSNIMNFFKTEITLANGEIR |
| | | KRPLIETNGETGEIVWDKGRDFATVRKVLSMPQVNIVKKTEVQTGGFSKESILPKRNSDKLIA |
| | | RKKDWDPKKYGGFDSPTVAYSVLVVAKVEKGKSKKLKSVKELLGITIMERSSFEKNPIDFLEAK |
| | | GYKEVKKDLIIKLPKYSLFELENGRKRMLASAGELQKGNELALPSKYVNFLYLASHYEKLKGSPE |
| | | DNEQKQLFVEQHKHYLDEIIEQISEFSKRVILADANLDKVLSAYNKHRDKPIREQAENIIHLFTL |
| | | TNLGAPAAFKYFDTTIDRKRYTSTKEVLDATLIHQSITGLYETRIDLSQLGGDPKKKRKVDPKKK |
| | | RKVDPKKKRKVGGGGSGGGGSGGGGSHHHHHH |
| 6 | C_ABD_Cas9_ | GPPKKKRKVDPKKKRKVDPKKKRKVMDKKYSIGLDIGTNSVGWAVITDEYKVPSKKFKVLGN |
| | External | TDRHSIKKNLIGALLFDSGETAEATRLKRTARRRYTRRKNRICYLQEIFSNEMAKVDDSFFHRL |
| | (SEQ ID | EESFLVEEDKKHERHPIFGNIVDEVAYHEKYPTIYHLRKKLVDSTDKADLRLIYLALAHMIKFRG |
| | NO: 28) | HFLIEGDLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQYADLFLAA |
| | | KNLSDAILLSDILRVNTEITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTFDNGSIPHQIHLGELHAIL |
| | | RRQEDFYPFLKDNREKIEKILTFRIPYYVGPLARGNSRFAWMTRKSEETITPWNFEEVVDKGA |
| | | SAQSFIERMTNFDKNLPNEKVLPKHSLLYEYFTVYNELTKVKYVTEGMRKPAFLSGEQKKAIV |
| | | DLLFKTNRKVTVKQLKEDYFKKIECFDSVEISGVEDRFNASLGTYHDLLKIIKDKDFLDNEENED |
| | | ILEDIVLTLTLFEDREMIEERLKTYAHLFDDKVMKQLKRRRYTGWGRLSRKLINGIRDKQSGKT |
| | | ILDFLKSDGFANRNFMQLIHDDSLTFKEDIQKAQVSGQGDSLHEHIANLAGSPAIKKGILQTV |
| | | KVVDELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLTRSDKNR |
| | | GKSDNVPSEEVVKKMKNYWRQLLNAKLITQRKFDNLTKAERGGLSELDKAGFIKRQLVETR |
| | | QITKHVAQILDSRMNTKYDENDKLIREVKVITLKSKLVSDFRKDFQFYKVREINNYHHAHDAY |
| | | LNAVVGTALIKKYPKLESEFVYGDYKVYDVRKMIAKSEQEIGKATAKYFFYSNIMNFFKTEITL |
| | | ANGEIRKRPLIETNGETGEIVWDKGRDFATVRKVLSMPQVNIVKKTEVQTGGFSKESILPKRN |
| | | SDKLIARKKDWDPKKYGGFDSPTVAYSVLVVAKVEKGKSKKLKSVKELLGITIMERSSFEKNPI |
| | | DFLEAKGYKEVKKDLIIKLPKYSLFELENGRKRMLASAGELQKGNELALPSKYVNFLYLASHYE |
| | | KLKGSPEDNEQKQLFVEQHKHYLDEIIEQISEFSKRVILADANLDKVLSAYNKHRDKPIREQAE |
| | | NIIHLFTLTNLGAPAAFKYFDTTIDRKRYTSTKEVLDATLIHQSITGLYETRIDLSQLGGDPKKKR |
| | | KVDPKKKRKVDPKKKRKVGSGGGGSGGGGSGGGGSLKEAKEKAIEELKKAGITSDYYFDLIN |
| | | KAKTVEGVNALKDEILKAHHHHHH |

Cas9 domain:
(SEQ ID NO: 29)
MDKKYSIGLDIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKRYDE
HHQDLTLLKALVRQQLPEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIEKILTFRIPYYVGPLARG
NSRFAWMTRKSEETITPWNFEEVVDKGASAQSFIERMTNFDKNLPNEKVLPKHSLLYEYFTVY
NELTKVKYVTEGMRKPAFLSGEQKKAIVDLLFKTNRKVTVKQLKEDYFKKIECFDSVEISGVEDR
FNASLGTYHDLLKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDHIVPQ
SFLKDDSIDNKVLTRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLSMPQVNIVKKTEVQTG
GFSKESILPKRNSDKLIARKKDWDPKKYGGFDSPTVAYSVLVVAKVEKGKSKKLKSVKELLGITI
MERSSFEKNPIDFLEAKGYKEVKKDLIIKLPKYSLFELENGRKRMLASAGELQKGNELALPSKYV
NFLYLASHYEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYETRIDLSQLG
GD

### ABD Sequences

1GJS or GA3:
(SEQ ID NO: 30)
LAEAKVLANRELDKYGVSDYYKNLINNAKTVEGVKALIDEILAALP
1TFO or ALB8-GA:
(SEQ ID NO: 31)
LKNAKEDAIAELKKAGITSDFYFNAINKAKTVEEVNALKNEILKAHA
2FS1 or PSD-1:
(SEQ ID NO: 32)
LAQAKEAAIKELKQYGIGDYYIKLINNAKTVEGVESLKNEILKA
ABDstable:
(SEQ ID NO: 33)
LAEAKVLALRELDKYGVSDYYKDLIDKAKTVEGVKALIDEILAA
ABD035:
(SEQ ID NO: 34)
LAEAKVLANRELDKYGVSDFYKRLINKAKTVEGVEALKLHILAALP
ABD094:
(SEQ ID NO: 35)
LAEAKEAANAELDSYGVSDFYKRLIDKAKTVEGVEALKDAILAALP

### NLS Sequences

Nucleoplasmin bipartite NLS:
(SEQ ID NO: 36)
KRPAATKKAGQAKKKK
L-MTAS:
(SEQ ID NO: 37)
GRYLTQETNKVETYKEQPLKTPGKKKKGKPGKRKEQEKKKRRTR
(SEQ ID NO: 38)
PKKKRKVDPKKKRKVDPKKKRKV

### Consensus NLS Sequences

(SEQ ID NO: 39)
KR(K/R)R, K(K/R)RK
(SEQ ID NO: 40)
(P/R)XXKR(^{∧}DE)(K/R)
(SEQ ID NO: 41)
KRX(W/F/Y)XXAF
(SEQ ID NO: 42)
(R/P)XXKR(K/R)(^{∧}DE)
(SEQ ID NO: 43)
KR(K/R)R, K(K/R)RK
(SEQ ID NO: 44)
(P/R)XXKR(^{∧}DE)(K/R)
(SEQ ID NO: 45)
KRX(W/F/Y)XXAF
(SEQ ID NO: 46)
(R/P)XXKR(K/R)(^{∧}DE)
Note:
(^{∧}DE) represents any amino acid except Asp or Glu. X represents any amino acid. Amino acids within parentheses [i.e. (K/R)] can represent either amino acid in that position. In other words, it can either be Lysine or it can be an Arginine.

### Linker Sequences

Flexible Linkers
(SEQ ID NO: 47)
(G)₈
(SEQ ID NO: 48)
(G)₉
(SEQ ID NO: 49)
KESGSVSSEQLAQFRSLD
(SEQ ID NO: 50)
EGKSSGSGSESKST
(SEQ ID NO: 51)
GSAGSAAGSGEF
Rigid Linkers
(SEQ ID NO: 52)
(EAAAK)₁₋₃
(SEQ ID NO: 53)
A(EAAAK)₄ALEA(EAAAK)₄A
(SEQ ID NO: 54)
PAPAP
(SEQ ID NO: 55)
A(EAAAK)₂A
Cleavable Linkers
(SEQ ID NO: 56)
GGGGSSPLGLWAGGGGS (MMP-1 sensitive)
(SEQ ID NO: 57)
TRHRQPRGWEQL (Furin sensitive)

What is claimed is:

1. A recombinant protein comprising:
an albumin binding domain;
a nuclear localization domain; and
a Cas9 domain.

2. The recombinant protein of claim 1, comprising a linker between the albumin binding domain and the nuclear localization domain.

3. The recombinant protein of claim 1, comprising more than one albumin binding domain, more than one nuclear localization domain, or a combination thereof.

4. The recombinant protein of claim 1, comprising 1 to 4 albumin binding domains.

5. The recombinant protein of claim 1, wherein the albumin binding domain comprises an amino acid sequence of SEQ ID NO:20.

6. The recombinant protein of claim 1, comprising 1 to 10 nuclear localization domains.

7. The recombinant protein of claim 1, wherein the nuclear localization domain comprises an amino acid sequence of SEQ ID NO:21.

8. The recombinant protein of claim 1, wherein the Cas9 domain comprises an amino acid sequence of SEQ ID NO:29.

9. The recombinant protein of claim 2, wherein the linker comprises an amino acid sequence of SEQ ID NO:22.

10. The recombinant protein of claim 1, wherein the albumin binding domain is between the Cas9 domain and the nuclear localization domain.

11. The recombinant protein of claim 2, comprising, in a N-terminus to a C-terminus direction:
the nuclear localization domain;
the linker;
the albumin binding domain; and
the Cas 9 domain.

12. The recombinant protein of claim 11, comprising an amino acid sequence that is at least 95% identical to any one of SEQ ID NO:26 and SEQ ID NO:27.

13. The recombinant protein of claim 12, comprising an amino acid sequence that is at least 99% identical to any one of SEQ ID NO:26 and SEQ ID NO:27.

14. The recombinant protein of claim 2, comprising, in a N-terminus to a C-terminus direction:
the nuclear localization domain;
the linker;
the albumin binding domain;
the Cas 9 domain;
the albumin binding domain;
the linker; and
the nuclear localization domain.

15. The recombinant protein of claim 14, comprising an amino acid sequence that is at least 95% identical to SEQ ID NO:26.

16. The recombinant protein of claim 15, comprising an amino acid sequence that is at least 99% identical to SEQ ID NO:26.

17. The recombinant protein of claim 1, having a molecular weight of about 100 kDa to about 300 kDa.

18. The recombinant protein of claim 1, having a binding affinity (K_{D}) to albumin of less than 50 nM.

19. The recombinant protein of claim 1, comprising an amino acid sequence that is at least 95% identical to any one of SEQ ID NO:24, SEQ ID NO:25, SEQ ID NO:26, SEQ ID NO: 27, and SEQ ID NO:28.

20. The recombinant protein of claim 19, comprising an amino acid sequence that is at least 99% identical to any one of SEQ ID NO:24, SEQ ID NO:25, SEQ ID NO:26, SEQ ID NO: 27, and SEQ ID NO:28.

21. A recombinant protein comprising a multi-domain structure of formula (I) 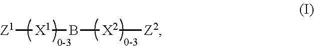
wherein:
B is a Cas9 domain;
X¹ and X², at each occurrence, are each independently a linker, an albumin binding domain, or a nuclear localization domain;
Z¹ and 22 are each independently an albumin binding domain or a nuclear localization domain, and
the multi-domain structure of formula (I) has at least one albumin binding domain and at least one nuclear localization domain.

22. The recombinant protein of claim 21, wherein the albumin binding domain comprises an amino acid sequence of SEQ ID NO:20.

23. The recombinant protein of claim 21, wherein the nuclear localization domain comprises an amino acid sequence of SEQ ID NO: 21.

24. The recombinant protein of claim 21, wherein the Cas9 domain comprises an amino acid sequence of SEQ ID NO:29.

25. The recombinant protein of claim 21, wherein the linker comprises an amino acid sequence of SEQ ID NO:22.

26. A composition comprising:
the recombinant protein of claim 1; and
one or more guide RNAs (gRNA).

27. The composition of claim 26, wherein the gRNA is a single-guide RNA (sgRNA), the sgRNA comprising a crRNA attached to a tracrRNA.

28. A method of modulating gene expression in a subject in need thereof, the method comprising administering to the subject a therapeutically effective amount of the composition of claim 26.

29. A method of treating a genetic disease in a patient in need thereof, the method comprising administering to the patient a therapeutically effective amount of a composition, the composition comprising:
a recombinant protein of claim 1; and
one or more gRNAs.

30. The method of claim 29, wherein the genetic disease is selected from any one of Duchenne muscular dystrophy (DMD), Limb Girdle muscular dystrophy (LGMD), myotonic dystrophy, Charcot-Marie Tooth Syndrome (CMT), Spinal Cerebellar Ataxia, and Huntington's disease.

31. A composition for use in the treatment of a genetic disease, the composition comprising:
a recombinant protein of claim 1; and
one or more gRNAs.

32. The composition for use of claim 31, wherein the genetic disease is selected from any one of Duchenne muscular dystrophy (DMD), Limb Girdle muscular dystrophy (LGMD), myotonic dystrophy, Charcot-Marie Tooth Syndrome (CMT), Spinal Cerebellar Ataxia, and Huntington's disease.

33. A method of modifying a targeted gene associated with disease in a patient, the method comprising administering to the patient a therapeutically effective amount of a composition, the composition comprising:
the recombinant protein of claim 1; and
one or more gRNAs.

34. The method of claim 33, wherein the targeted gene is selected from any one of a dystrophin (DMD) gene, a calpain 3 (CAPN3) gene, a dysferlin (DYSF) gene, an alpha-sarcoglycan (SGCA) gene, a beta-sarcoglycan (SGCB) gene, a gamma-sarcoglycan (SGCG) gene, a delta-sarcoglycan gene (SGCD) gene, a telethonin (TCAP) gene, a fukutin-related protein (FKRP) gene, an anoctamin 5 (ANO5) gene, a DM1 gene, a DM2 gene, a neurotrophin 3 (NT3) gene, and a huntingtin (HTT) gene.

35. The method of claim 34, wherein the targeted gene is a human gene.
